(19) United States
(12) Patent Application Publication
(10) Pub. No.: US 2017/0030890 A1
(43) Pub. Date: Feb. 2, 2017

(54) MICROFILTRATION DEVICE

(71) Applicant: Infineon Technologies AG, Neubiberg (DE)

(72) Inventors: Gerald Holweg, Graz (AT); Yonsuang Arnanthigo, Villach (AT); Jan Berger, Villach (AT); Guenter Denifl, Annenheim (AT); Sylvicley Figueira Da Silva, Villach (AT); Iris Moder, Villach (AT); Thomas Ostermann, Velden am Woerthersee (AT); Alexander Oswatitsch, Villach-Landskron (AT); Vijaye Kumar Rajaraman, Villach (AT); Gudrun Stranzl, Goedersdorf (AT)

(21) Appl. No.: 15/221,010

(22) Filed: Jul. 27, 2016

(30) Foreign Application Priority Data
Jul. 31, 2015 (DE) 10 2015 112 603.6

(51) Int. Cl. G01N 33/49; B01D 69/02; B01D 71/02; B01D 61/14

(52) U.S. Cl. CPC G01N 33/491; B01D 61/147; B01D 69/02; B01D 71/02; B01D 71/022; B01D 71/027; B01D 2325/02; B01D 2325/26

(57) ABSTRACT

A microfiltration device comprises a substrate having a first surface and a second surface opposite to the first surface. The substrate includes a cavity between the first surface and the second surface. The substrate further includes a microfilter including a frame part in contact with the substrate and a filter part abutting the cavity. The microfilter comprises in both the frame part and the filter part a semiconducting or conducting material.

### CROSS REFERENCE TO RELATED APPLICATIONS

This application claims priority to German Application No. 10 2015 112 603.6, filed on Jul. 31, 2015, and incorporated herein by reference in its entirety.

### BACKGROUND

Blood tests are carried out on patients to determine various diseases and body condition. Blood-plasma for instance carries information on glucose, salts, hormones, blood-gas, for example. An overview of the components constituting blood, in particular human blood, is shown in FIG. 31. Blood comprises two main components. On the one hand, blood contains blood plasma, which contains plasma proteins such as albumins, globulins or fibrinogen (clotting proteins) and serum such as water, salts, dissolved gases, hormones, glucose and wastes. On the other hand, blood contains cellular components such as platelets having a size in a range of 1 to 2 μm, red blood cells having a size around 7 μm or white blood cells (leukocytes). The white blood cells comprise granular leukocytes and agranular leukocytes. The granular leukocytes comprise neutrophil leukocytes having a size between 10 to 14 μm, eosinophil leukocytes having a size between 10 to 14 μm and basophil leukocytes having a size between 10 to 14 μm. The agranular leukocytes comprise monocytes having a size between 15 to 20 μm and lymphocytes having a size between 8 to 10 μm. Blood-plasma as well as white blood cells present in whole-blood carries also information on infection. Today, blood-plasma is extracted using clinical procedure involving centrifugation, which is manual, laborious and time-consuming. Further, this procedure cannot be administered at home by patients.

It is thus desirable to provide a device enabling patients to measure at least one blood-related parameter at their homes instead of clinical/laboratory-based testing.

### SUMMARY

According to an embodiment of a microfiltration device, a microfiltration device comprises a substrate having a first surface and a second surface opposite to the first surface. The substrate includes a cavity between the first surface and the second surface. The substrate further includes a microfilter including a frame part in contact with the substrate and a filter part abutting the cavity. The microfilter comprises in both the frame part and the filter part a semiconducting or conducting material.

According to an embodiment of a sensor device, the sensor device comprises the microfiltration device and a sensor located at a filtrate side of the filter part. The sensor is adapted to measure a characteristic of a filtrate.

According to an embodiment of a multisensor device, the multisensor device comprises at least two sensor devices arranged next to each other in a lateral direction.

According to another embodiment of a multisensor device, the multisensor device comprises at least two sensor devices stacked on each other in a vertical direction.

Those skilled in the art will recognize additional features and advantages upon reading the following detailed description and on viewing the accompanying drawings.

### BRIEF DESCRIPTION OF THE DRAWINGS

The accompanying drawings are included to provide a further understanding of embodiments of the invention and are incorporated in and constitute a part of this specification. The drawings illustrate the embodiments of the present invention and together with the description serve to explain the principles. Other embodiments of the invention and many of the intended advantages will be readily appreciated, as they become better understood by reference to the following detailed description. The elements of the drawings are not necessarily to scale relative to each other. Like reference numbers designate corresponding similar parts.

FIG. 1 is a schematic view of a microfiltration device according to an embodiment.

FIG. 2A is a schematic view of a sensor device according to an embodiment.

FIG. 2B is a schematic view of a multisensor device according to an embodiment.

FIG. 2C is a schematic view of a multisensor device according to another embodiment.

FIGS. 3A to 3G are cross-sectional views illustrating a method of manufacturing a microfiltration device of FIG. 3H.

FIG. 3H is a schematic cross-sectional view of a portion of a microfiltration device according to an embodiment.

FIGS. 4A to 4H are cross-sectional views illustrating a method for manufacturing a microfiltration device of FIG. 4I.

FIG. 4I is a schematic cross-sectional view of a portion of a microfiltration device according to an embodiment.

FIGS. 5A to 5F are cross-sectional views illustrating a method for manufacturing a microfiltration device of FIG. 5G.

FIG. 5G is a schematic cross-sectional view of a portion of a microfiltration device according to an embodiment.

FIG. 6 is a schematic cross-sectional view of a portion of a microfiltration device according to an embodiment.

FIGS. 7A to 7D are cross-sectional views illustrating a method for manufacturing a microfiltration device of FIG. 7E.

FIG. 7E is a schematic cross-sectional view of a portion of a microfiltration device according to an embodiment.

FIGS. 8A to 8F are cross-sectional views illustrating a method for manufacturing a microfiltration device of FIG. 8G.

FIG. 8G is a schematic cross-sectional view of a portion of a microfiltration device according to an embodiment.

FIGS. 9A to 9D are cross-sectional views illustrating a method for manufacturing a microfiltration device of FIG. 9E.

FIG. 9E is a schematic cross-sectional view of a portion of a microfiltration device according to an embodiment.

FIGS. 10A to 10D are cross-sectional views illustrating a method for manufacturing a microfiltration device of FIG. 10.

FIG. 10E is a schematic cross-sectional view of a portion of a microfiltration device according to an embodiment.

FIG. 11 is a schematic cross-sectional view of a portion of a microfiltration device according to an embodiment.

FIG. 12A to 12H are cross-sectional views illustrating a method for manufacturing a microfiltration device of FIG. 12I.

FIG. 12I is a schematic cross-sectional view of a portion of a microfiltration device according to an embodiment.

FIGS. 13A to 13G are cross-sectional views illustrating a method for manufacturing a microfiltration device of FIG. 13H.

FIG. 13H is a schematic cross-sectional view of a portion of a microfiltration device according to an embodiment.

FIG. 14 to FIG. 20 are schematic cross-sectional views of a portion of a sensor device according to different embodiments.

FIG. 21 is a schematic perspective view of a microfiltration device employed in the sensor devices of FIG. 19 and FIG. 20.

FIG. 22 is a cross-sectional view of the microfiltration device taken along the section plane A-A′ of FIG. 21.

FIG. 23 is a cross-sectional view of the micro-filtration device comprising supporting pillars taken along the section plane A-A′ of FIG. 21.

FIG. 24 is a plan view of the microfiltration device of FIG. 21 comprising supporting pillars.

FIGS. 25 and 26 are schematic cross-sectional views of a sensor device according to different embodiments.

FIG. 27 is a plan view of a multisensor device according to an embodiment.

FIG. 28 is a schematic cross-sectional view taken along the section plane B-B′ of FIG. 27.

FIG. 29 is a schematic cross-sectional view taken along the section plane C-C′ of FIG. 27.

FIG. 30 is a schematic cross-sectional view of a multisensor device according to another embodiment.

FIG. 31 is a schematic diagram illustrating components of blood.

FIG. 32 is a diagram illustrating a dependency of a gap between electrodes and their applied voltage for micro-dripping mode of blood.

FIG. 33A and FIG. 33B are schematic cross-sectional views of a portion of a sensor device according to further embodiments.

FIG. 34A and FIG. 34B are plan views of a sensor device according to different embodiments.

### DETAILED DESCRIPTION

In the following detailed description reference is made to the accompanying drawings, which form a part hereof and in which are illustrated by way of illustration specific embodiments in which the invention may be practiced. In this regard, directional terminology such as “top”, “bottom”, “front”, “back”, “leading”, “trailing” etc. is used with reference to the orientation of the Figures being described. Since components of embodiments of the invention can be positioned in a number of different orientations, the directional terminology is used for purposes of illustration and is in no way limiting. It is to be understood that other embodiments may be utilized and structural or logical changes may be made without departing from the scope defined by the claims.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The Figures and the description illustrate relative doping concentrations by indicating “−” or “+” next to the doping type “n” or “p”. For example, “n⁻” means a doping concentration which is lower than the doping concentration of an “n”-doping region while an “n⁺”-doping region has a higher doping concentration than an “n”-doping region. Doping regions of the same relative doping concentration do not necessarily have the same absolute doping concentration. For example, two different “n”-doping regions may have the same or different absolute doping concentrations. In the Figures and the description, for the sake of a better comprehension, often the doped portions are designated as being “p” or “n”-doped. As is clearly to be understood, this designation is by no means intended to be limiting. The doping type can be arbitrary as long as the described functionality is achieved. Further, in all embodiments, the doping types can be reversed.

As employed in this specification, the terms “coupled” and/or “electrically coupled” are not meant to mean that the elements must be directly coupled together—intervening elements may be provided between the “coupled” or “electrically coupled” elements. The term “electrically connected” intends to describe a low-ohmic electric connection between the elements electrically connected together.

The present specification refers to a “first” and a “second” conductivity type of dopants, semiconductor portions are doped with. The first conductivity type may be p type and the second conductivity type may be n type or vice versa. As is to be clearly understood, within the context of the present specification, the doping types may be reversed. If a specific current path is described using directional language, this description is to be merely understood to indicate the path and not the polarity of the current flow. The Figures may include polarity-sensitive components, e.g. diodes. As is to be clearly understood, the specific arrangement of these polarity-sensitive components is given as an example and may be inverted in order to achieve the described functionality, depending on whether the first conductivity type means n-type or p-type.

The terms “lateral” and “horizontal” as used in this specification intends to describe an orientation parallel to a first surface of a substrate or semiconductor body. This can be for instance the surface of a wafer or a die.

The term “vertical” as used in this specification intends to describe an orientation which is arranged perpendicular to the first surface of the semiconductor substrate or semiconductor body.

The terms “wafer”, “substrate” or “semiconductor body” used in the following description may include any semiconductor-based structure that has a semiconductor surface. Wafer and structure are to be understood to include silicon, silicon-on-insulator (SOI), silicon-on-sapphire (SOS), doped and undoped semiconductors, epitaxial layers of silicon supported by a base semiconductor foundation, and other semiconductor structures. The semiconductor need not be silicon-based. The semiconductor could as well be silicon-germanium, germanium, or gallium arsenide. According to other embodiments, silicon carbide (SiC) or gallium nitride (GaN) may form the semiconductor substrate material.

It is to be understood that the features of the various embodiments described herein may be combined with each other, unless specifically noted otherwise.

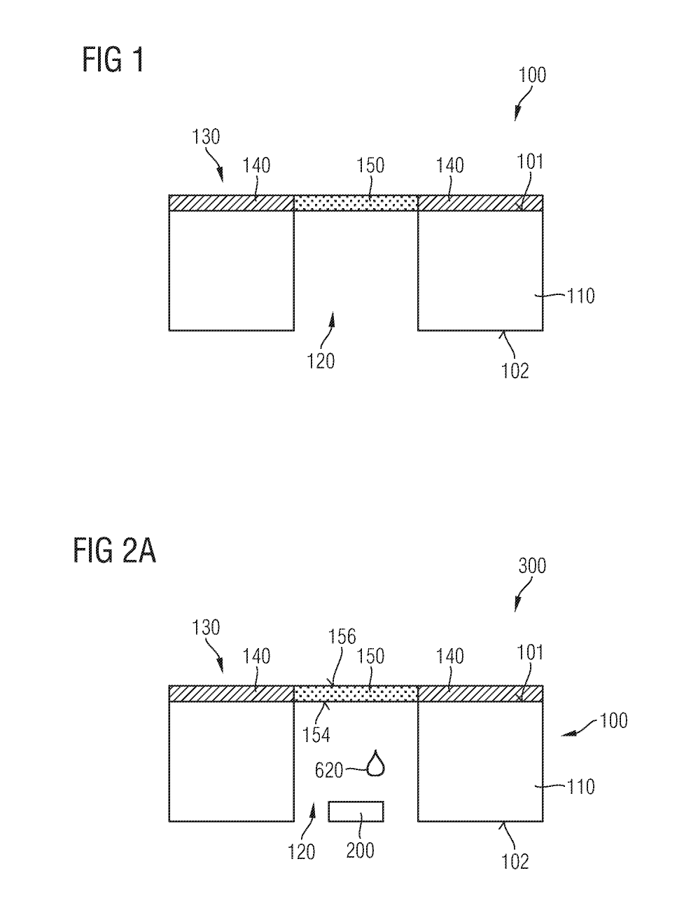

FIG. 1 is a schematic view of a microfiltration device 100 according to an embodiment.

As can be seen from FIG. 1, the microfiltration device 100 comprises a substrate 110. The substrate 110 has a first surface 101 and a second surface 102 opposite to the first surface 101. The substrate 110 includes a cavity 120 between the first surface 101 and the second surface 102. The microfiltration device 100 further comprises a microfilter 130. The microfilter 130 includes a frame part 140 in contact with the substrate 110 and a filter part 150 abutting the cavity 120. The microfilter 130 comprises in both the frame part 140 and the filter part 150 a semiconducting or conducting material.

By providing a microfilter 130 comprising in both the frame part 140 and the filter part 150 a semiconducting or conducting material, a filter part 150 having a tunable stiffness may be provided, which is further elastic and robust compared to filter parts of an insulating material such as a silicon nitride membrane or a polymer membrane. Further, microfiltration devices using polymer membranes are thick (about 330 microns), large, slow and hence require application of pressure for blood filtration. Microfiltration devices using silicon nitride monolayer membranes on the other hand are too thick to manufacture in a standard semiconductor manufacturing fab and it may also suffer from low robustness.

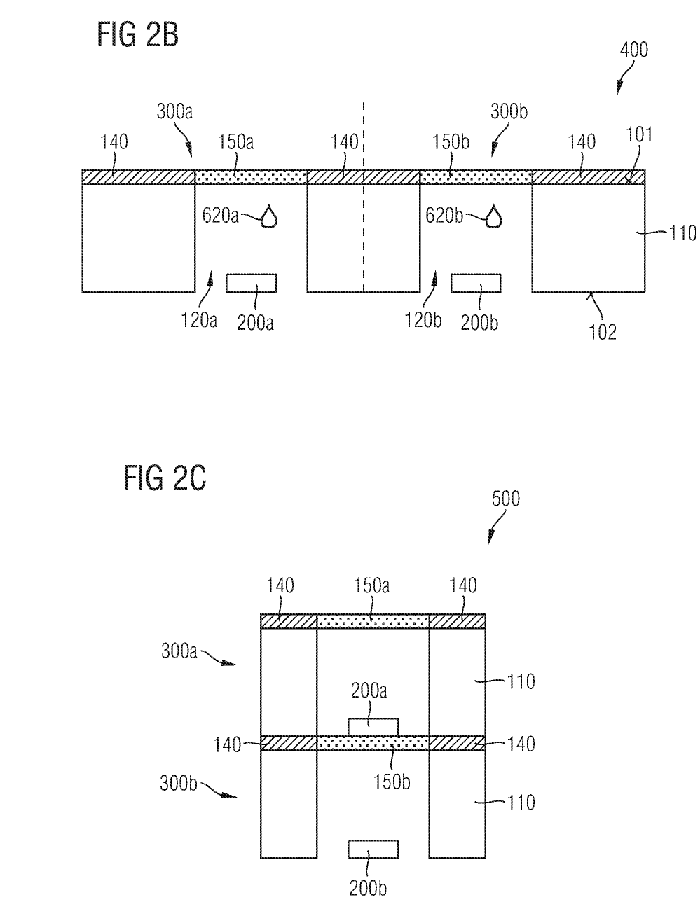

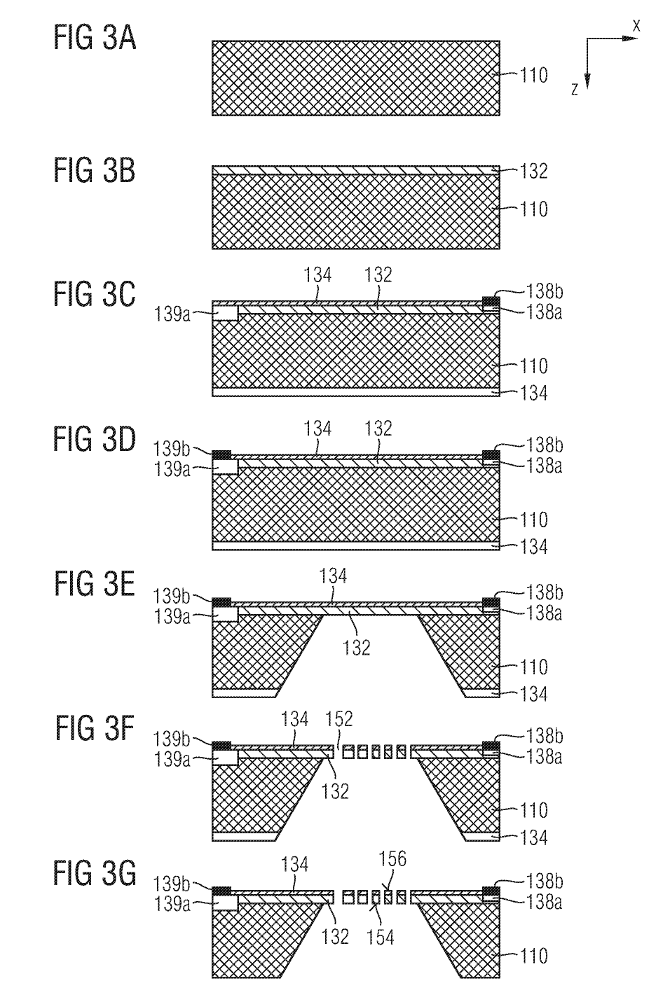

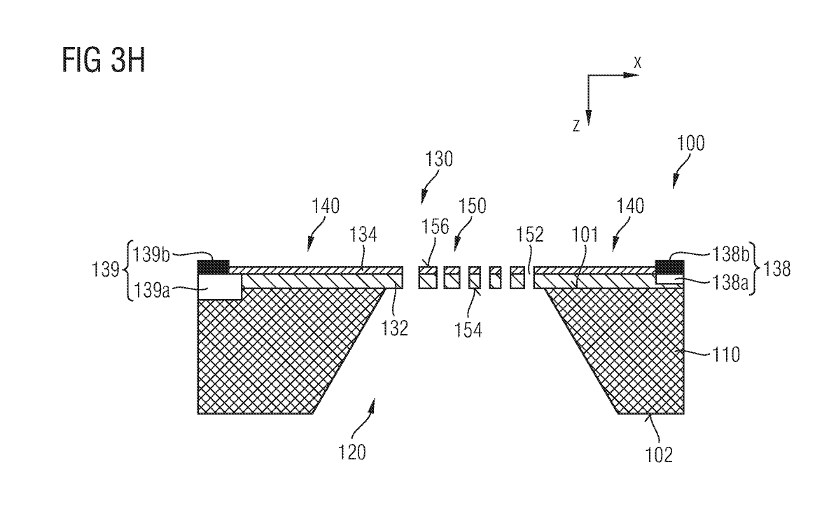

FIG. 2A is a schematic view of a sensor device 300 according to an embodiment. As can be seen from FIG. 2A, a sensor device 300 comprises the microfiltration device 100 according to an embodiment. Furthermore, the sensor device 300 comprises a sensor 200 located at a filtrate side 154 of the filter part 150. The sensor 200 is adapted to measure a characteristic of a filtrate 620. The filtrate 620 may be introduced into the cavity 120 along a direction extending from the first surface 101 to the second surface 102, wherein the filtrate side 154 is located on the same side as the second surface 102. In this case, a feed side 156 is located at the same side as the first surface 101 of the substrate 110. However, the filtrate 620 may also be introduced into a measurement chamber 325 through the filter part 150 along a direction extending from the second surface 102 of the first surface 101 of the substrate 110. In this case, the feed side 156 and the filtrate side 154 have to be interchanged. Such an embodiment is illustrated, for example, in FIG. 16.

FIG. 2B is a schematic view of a multisensor device 400 according to an embodiment. As shown in FIG. 2B, at least two sensor devices 300a, 300b are arranged next to each other in a lateral direction. As can be further seen from FIG. 2B, the multisensor device 400 comprises two sensors 200a, 200b, which are arranged to measure respective characteristics of filtrates 620a and 620b, which are introduced into respective cavities 120a, 120b through filter parts 150a, 150b. The filtration properties of the filter parts 150a and 150b may be different such that the sensors 200a and 200b measure the characteristic of different filtrates 620a and 620b.

FIG. 2C is a schematic view of a multisensor device 500 according to another embodiment. As shown in FIG. 2C, the multisensor device 500 comprises at least two sensor devices stacked on each other in a vertical direction. As can be further seen from FIG. 2C, the multisensor device 500 comprises sensors 200a and 200b. The sensor 200b is arranged in a downstream part of a filtrate flow direction. By providing a plurality of sensor devices 300a, 300b stacked on each other in a vertical direction, a cascaded filtration by the filter parts 150a and 150b may be achieved. Herein, the pore size of the filter parts 150a, 150b may be reduced along the filtrate flow direction.

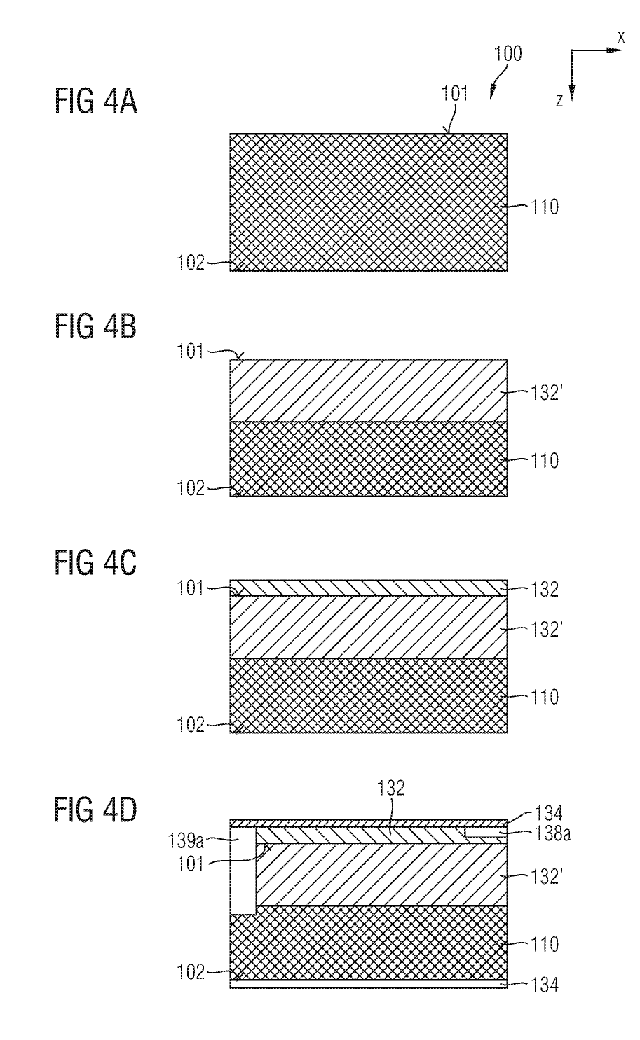

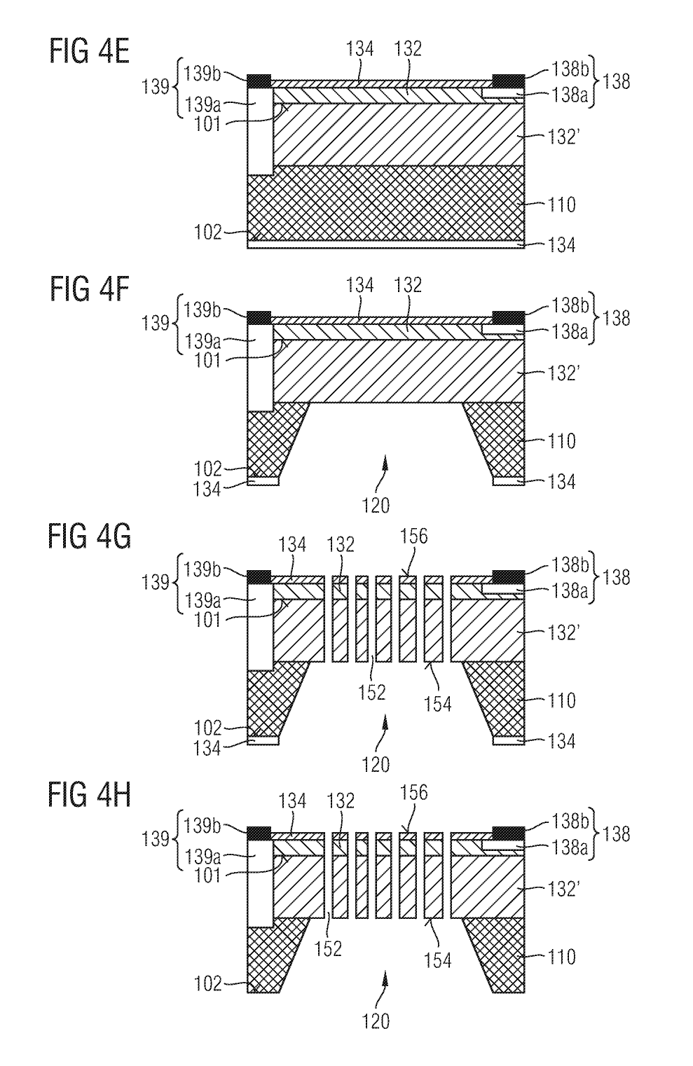

FIG. 3H is a schematic cross-sectional view of a portion of a microfiltration device 100 according to an embodiment. As can be seen from FIG. 3H, the microfiltration device 100 comprises the substrate 110, on which the microfilter 130 is formed. The microfilter 130 comprises a first layer 132 of the semiconducting or conducting material and a second layer 134 of an insulating material. The substrate 110 may include an insulating material. The substrate 110 may, however, also include a semiconducting or conducting material.

The substrate 110 may be semiconductor body, which may be provided form a single-crystalline semiconductor material, for example silicon Si, silicon carbide SiC, germanium Ge, a silicon germanium crystal SiGe, gallium nitride GaN or gallium arsenide GaAs. A distance between the first and second surfaces 101, 102 may be at least 20 μm, for example at least 50 μm. Other embodiments may provide semiconductor bodies with a thickness of several 100 μm. The substrate 110 may have a rectangular shape with an edge length in the range of several millimetres.

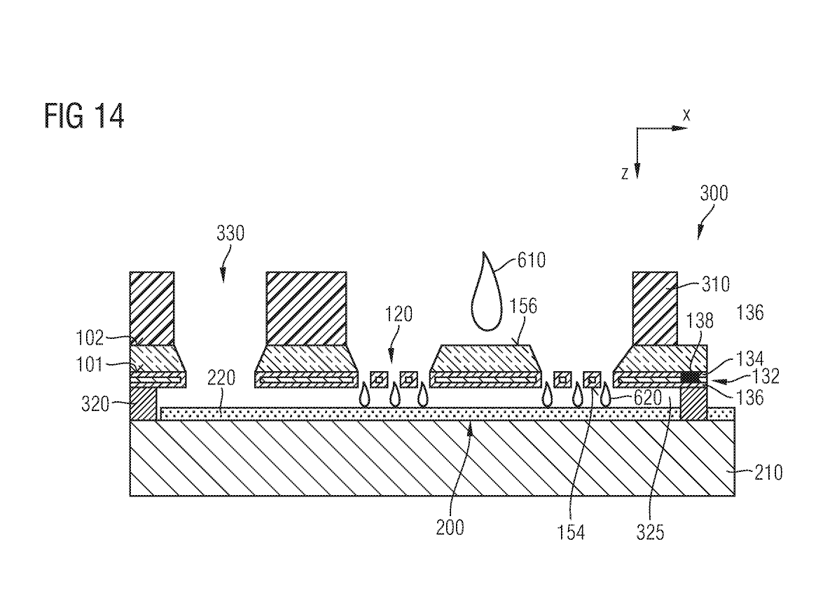

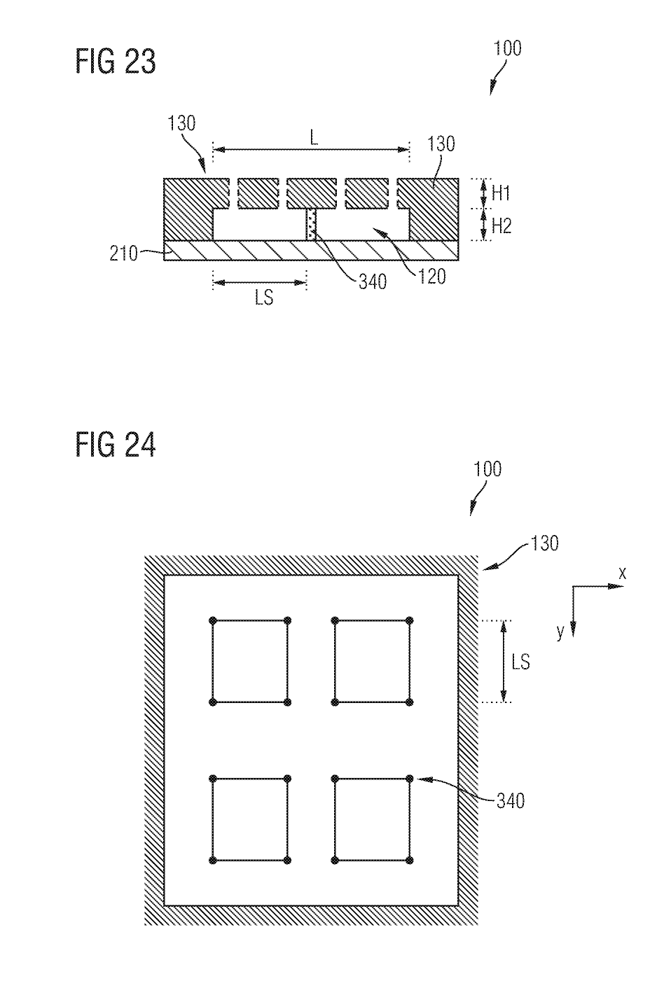

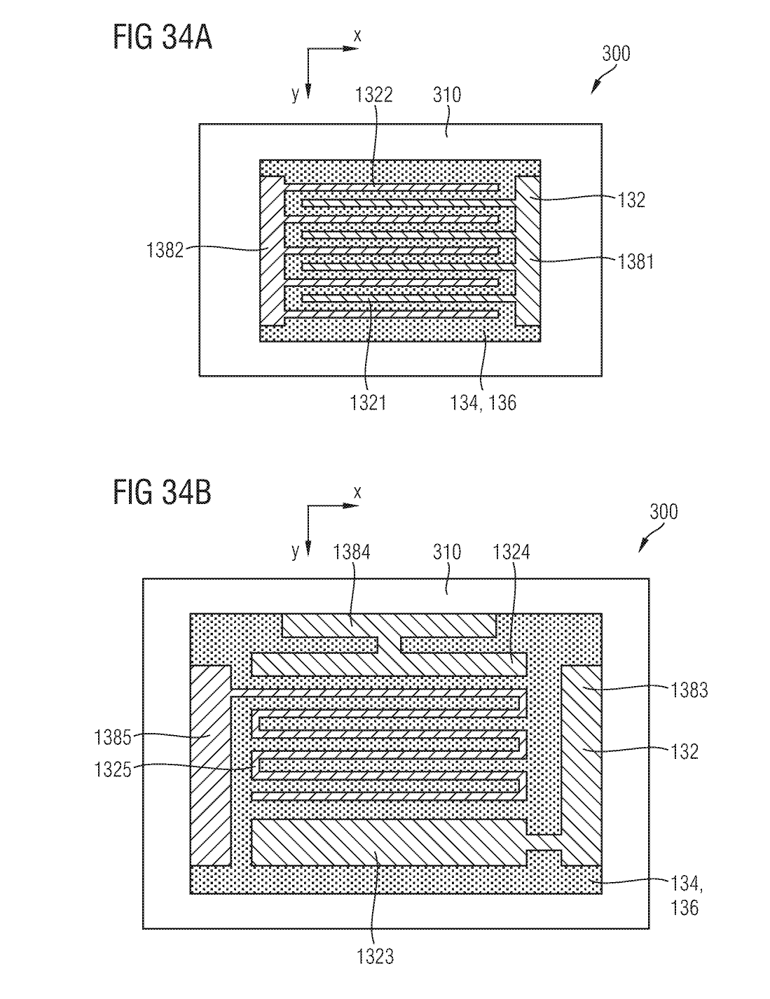

The normal to the first and second surfaces 101, 102 defines the vertical direction z and directions orthogonal to the normal direction are first and second lateral directions x, y (cf. also FIG. 21, FIG. 27, and FIGS. 34A and 34B).

The semiconducting or conducting material may comprise at least one of a polysilicon, silicon, porous silicon, and a metal. The semiconducting or conducting material may further comprise germanium Ge, a silicon germanium SiGe, gallium nitride GaN or gallium arsenide GaAs. The semiconducting or conducting material may comprise a semiconductor material of an n-type or a p-type. The semiconducting or conducting material may further comprise a metal material, which may consist of or contain, as main constituent(s) aluminium Al, copper Cu or alloys of aluminium or copper, for example AlSi, AlCu, or AlSiCu. According to other embodiments, the metal material may contain one, two, three or more sub-layers, each sub-layer containing, as a main constituent, at least one of nickel Ni, titanium Ti, silver Ag, gold Au, tungsten W, platinum Pt, tantalum Ta and palladium Pd. For example, a sub-layer may contain a metal nitride or a metal alloy containing Ni, Ti, Ag, Au, W, Pt, Co and/or Pd.

The insulating material may comprise at least one of an oxide, a nitride, a carbide, and a glass. In particular, the insulating material may comprise silicon oxide, silicon nitride, or silicon carbide. The insulating material may further comprise a tetraethylorthosilicate (TEOS)/undoped silicate glass (USG) film. The insulating material may further comprise a phosphosilicate glass (PSG) or a borophosphosilicate glass (BPSG). The insulating material may further include one or any combination of an oxide, nitride, oxynitride, a high-k material, an imide, and insulating resin or glass, for example.

According to the embodiment of FIG. 3H, the first layer 132 may be an epitaxial layer of a semiconductor such as silicon of a first conductivity type, for example an n-type. In order to electrically insulate the conducting epitaxial silicon layer of a first conductivity type from the substrate 110, the substrate 110 may be a semiconductor body of a second conductivity type, for example a p-type. On the first layer 132, a second layer 134 is formed, which may be of an insulating material. According to an embodiment, the second layer 134 may be an oxide layer, such as an silicon oxide layer.

In the filter part 150, pores 152 are formed, which are extended through the second layer 134 and the first layer 136 from a feed side 156 to a filtrate side 154.

The microfiltration device 100 further may comprise a contact area 138 in the frame part 140. The contact area 138 may be electrically connected to the semiconducting or conducting material in the filter part 150. According to the embodiment of FIG. 3H, the contact area 138 comprises a contact zone 138a, which may be of a first conductivity type, for example an n+-doped zone in the first layer 132, and further comprises a contact pad 138b, which may comprise a metal. According to an embodiment, a contact area 138 may also only comprise the contact pad 138b for contacting the semiconducting or conducting material in the first layer 132 in the filter part 150.

In addition, a substrate contact area 139 is provided in the frame part 140, which is electrically connected to the substrate 110 of a second conductivity type. The substrate contact area 139 comprises a substrate contact zone 139a, which may be of a second conductivity type, for example an p+-doped zone in the first layer 132 and the substrate 110, and further comprises a substrate contact pad 139b, which may comprise a metal.

The microfiltration device 100 thus comprises the contact area 138 being electrically connected to the semiconducting or conducting material in the first layer 132 of the filter part 150. In addition, the substrate contact area 139 is provided, which is electrically connected to the substrate 110 of a second conductivity type. Thus, the substrate 110 of a second conductivity type can be electrically insulated from the first layer 132 of a first conductivity type by generating a junction barrier between the substrate 110 and the first layer 132, if respective voltages are applied between the contact area 138 and the substrate contact area 139.

FIGS. 3A to 3G are cross-sectional views illustrating a method of manufacturing the microfiltration device of FIG. 3H. As can be seen from FIGS. 3A and 3B, a substrate 110 is provided, on which the first layer 132 is formed, e.g. by epitaxial growth or deposition. As can be seen from FIG. 3C, the second layer 134 of an insulating material is formed on the first layer 132. The second layer 134 may include any of the insulating material as discussed above or any dielectric adapted to isolate the first layer 132 from a surrounding environment.

The second layer 134 may, for example, include a field dielectric such as a field oxide and/or a gate dielectric such as a gate oxide. The second layer 134 may include a field oxide formed e.g. by a local oxidation of silicon (LOCOS process), deposited oxide or STI (shallow trench isolation). The thickness of the field dielectric of the second layer 134 may be in a range of 0.06 μm to 5 μm or 0.1 μm to 3 μm, the thickness of the gate dielectric of the second layer 134 may be in a range of 5 nm to 200 nm or 40 nm to 120 nm. The second layer 134 may also comprise a stack of different dielectric layers. According to the embodiment of FIG. 3C, the second layer 134 is formed as a thermal silicon oxide layer having a thickness in a range of 5 nm to 500 nm.

In addition, as can be seen from FIG. 3C, the contact zone 138a is formed in the first layer 132 by an implantation of dopants of a first conductivity type such as a n-type. Further, the substrate contact zone 139a is formed in the first layer 132 and the substrate 110 by an implantation of dopants of a second conductivity type such as a p-type.

As can be seen from FIG. 3D, the contact pad 138b of the contact area 138 is formed on the contact zone 138a by removing the insulating material of the second layer 134 at the contact zone 138a and by depositing a metal to form the contact pad 138b. In addition, the substrate contact pad 139b of the substrate contact area 139 is formed on the substrate contact zone 139a by removing the insulating material of the second layer 134 at the substrate contact zone 139a and by depositing a metal to form the substrate contact pad 139b.

As can be seen from FIG. 3C to FIG. 3E, the second layer 134 is formed also on the second surface 102 of the substrate 110. The second layer 134 is used as a etching mask layer for forming the cavity 120 in the substrate 110 in FIG. 3E, and is used as a etching mask layer to form the pores 152 in the filter part 150 in FIG. 3F. The cavity 120 and the pores 152 may be formed by an electro-chemical wet-etching process using wet etchant-solutions such as KOH, tetramethylammonium hydroxide (TMAH), for example.

As can be seen from FIG. 3G, the second layer 134 on the backside is removed from the second surface 102 to form the microfiltration device 100 as shown in FIG. 3H.

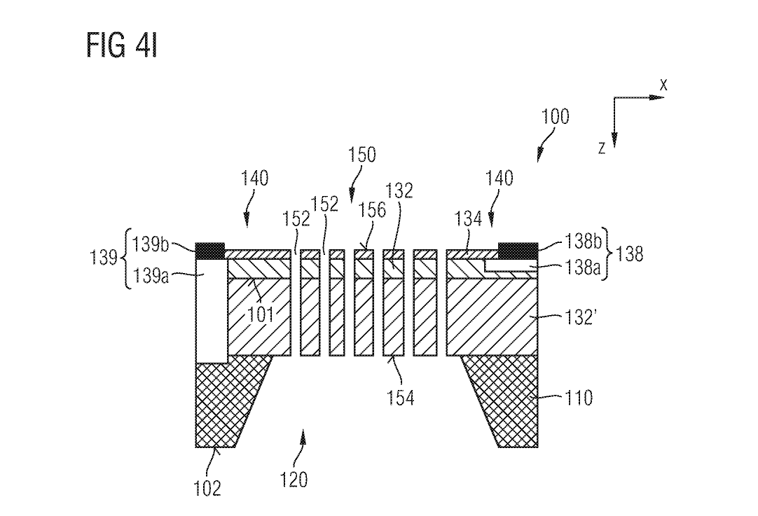

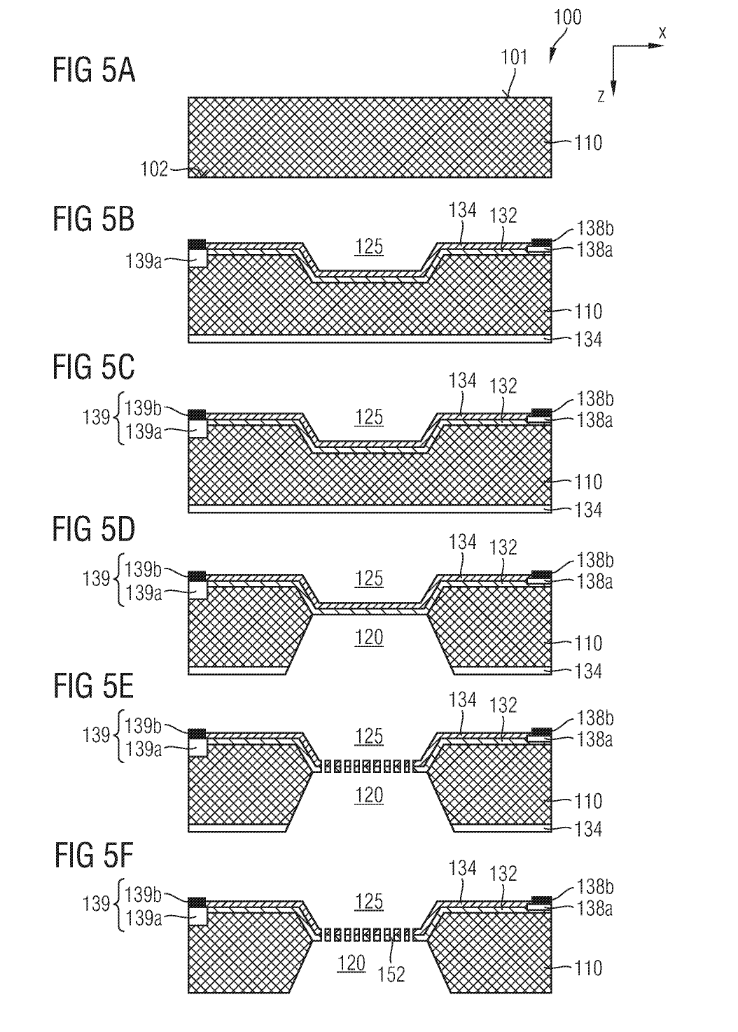

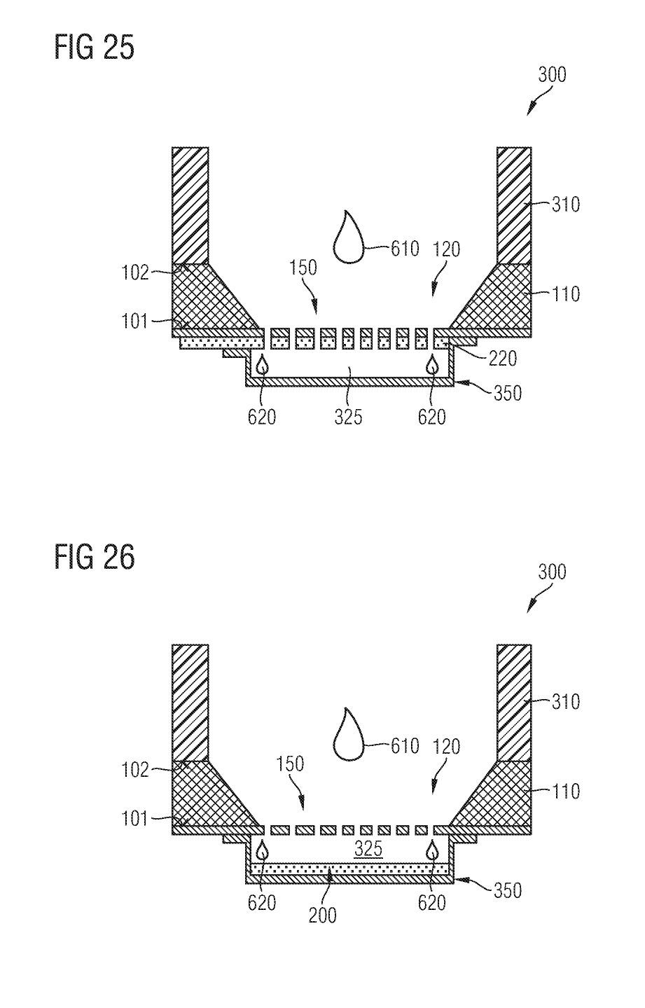

FIG. 4I shows another embodiment of the microfiltration device 100. As can be seen from comparing FIGS. 3H and 4I, in the milcrofiltration device 100 of FIG. 4I, the first layer 132 is formed on a substrate part 132′ of a first conductivity type. Thus, the substrate part 132′ and the first layer 132, which are both of a first conductivity type, form a layer of a semiconducting or conducting material. Since the epitaxially grown first layer 132 is further supported by the substrate part 132′, the filter part 150 can be formed with a higher thickness leading to higher robustness of the microfiltration device 100. The thickness of the filter part 150 in a vertical direction (ortohogonal to the first surface 101) may be in a range of 100 nm to 30 μm, or in a range of 1 μm to 20 μm, or in a range of 1 μm to 20 μm, the thickness of the frame part 140 in a vertical direction (ortohogonal to the first surface 101) including the substrate 110 may be in a range of 30 μm to 1000 μm, or in a range of 100 μm to 800 μm, or in a range of 500 μm to 800 μm. In addition, pores 152 having a greater pore size than the pores 152 in the microfiltration device 100 of FIG. 3H can be formed in the stacked layer of the first layer 132 and the substrate part 132′. As can be seen from FIGS. 27 and 28, and as will be discussed later in all detail, the microfiltration device 100 of FIG. 3H and the microfiltration device 100 of FIG. 4I can be manufactured in one process for forming a multisensor device 400 having filter parts 150 with different pore sizes.

As already discussed with regard to the microfiltration device 100 of FIG. 3H, the microfiltration device of FIG. 4I comprises also the contact zone 138a and the contact pad 138b, to form the contact area 138. Furthermore, the microfiltration device 100 of FIG. 4I comprises also the substrate contact zone 139a and the substrate contact pad 139b, to form the substrate contact area 139.

FIG. 4A to 4H are schematic cross-sectional views illustrating a method of manufacturing the microfiltration device of FIG. 4I.

As shown in FIGS. 4A and 4B, a substrate 110 is provided, wherein the substrate part 132′ is formed in the substrate 110 of the second conductivity type by an implantation process of dopants of a first conductivity type such as an n-type, followed by a thermal diffusion process.

As can be seen from FIG. 4C, the first layer 132 is formed on the substrate part 132′, e.g. by epitaxial growth or deposition.

As can be seen from FIGS. 4D and 4E, the second layer 134 is formed on the first layer 132 and the second surface 102 of the substrate 110. Furthermore, the contact area 138 and the substrate contact area 139 are formed as described above with regard to FIGS. 3C and 3D, wherein the substrate contact area 139 is extended to the substrate 110 of the second conductivity type.

As can be seen from FIGS. 4F and 4G, the second layer 134 is patterned by an lithographical process to be used as an etch mask layer to form the cavity 120 (FIG. 4F) and the pores 152 in the filter part 150 (FIG. 4G). The etching process includes an electro-chemical wet-etching process. By removing the second layer 134 on the second surface 102 of the substrate 110 (FIG. 4H), the microfiltration device 100 of FIG. 4I is provided.

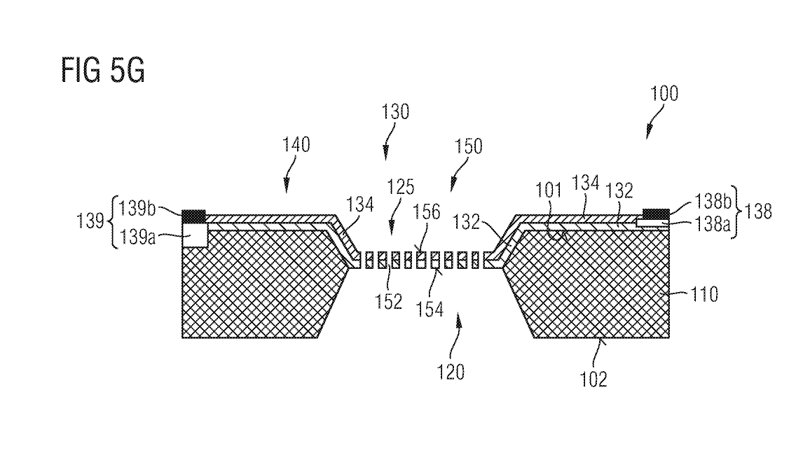

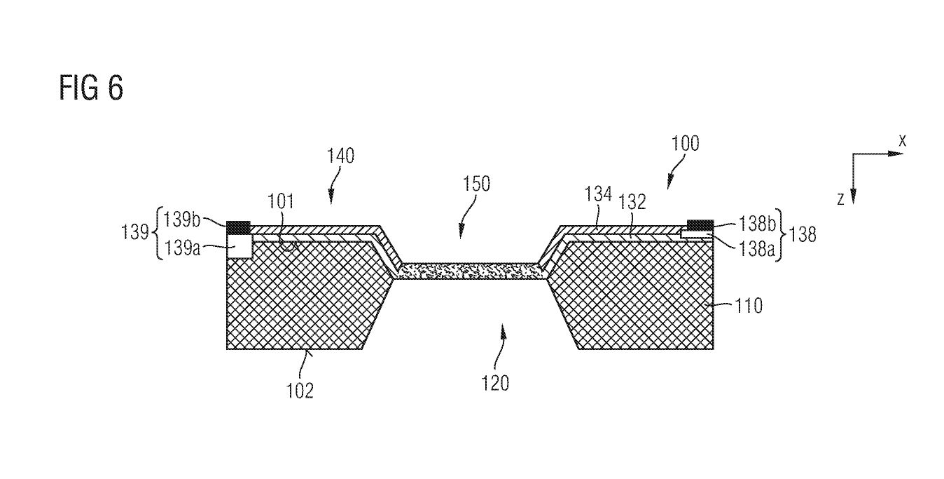

FIG. 5G shows a schematic cross-sectional view of a portion of a microfiltration device 100 according to another embodiment. As can be further seen from FIG. 5G, the filter part 150 of the microfilter 130 is not flush with the frame part 140 of the microfilter 130, but is located between the cavity 120 and an additional cavity 125.

FIG. 5A to 5F are cross-sectional views illustrating a method for manufacturing the microfiltration device of FIG. 5G.

As can be seen from FIGS. 5A and 5B, a substrate 110 is provided, in which the additional cavity 125 is formed. Thereafter, the first layer 132 is formed, e.g. by epitaxial growth or deposition. After forming a contact zone 138a by implanting dopants of a first conductivity type and forming a substrate contact zone 139a by implanting dopants of a second conductivity type, the second layer 134 is formed on the first layer 132.

As shown in FIG. 5C, the contact pad 138b and the substrate contact pad 139b are formed after removing a part of the second layer 134 covering the contact zone 138a and the substrate contact zone 139a, respectively.

As shown in FIGS. 5D and 5E, the second layer 134 is lithographically patterned and the cavity 120 (FIG. 5D) and the pores 152 (FIG. 5E) are formed by an electro-chemical wet-etching process. After removing the second layer 134 on the second surface 102 of the substrate 110 (FIG. 5F), the microfiltration device 100 of FIG. 5G is provided.

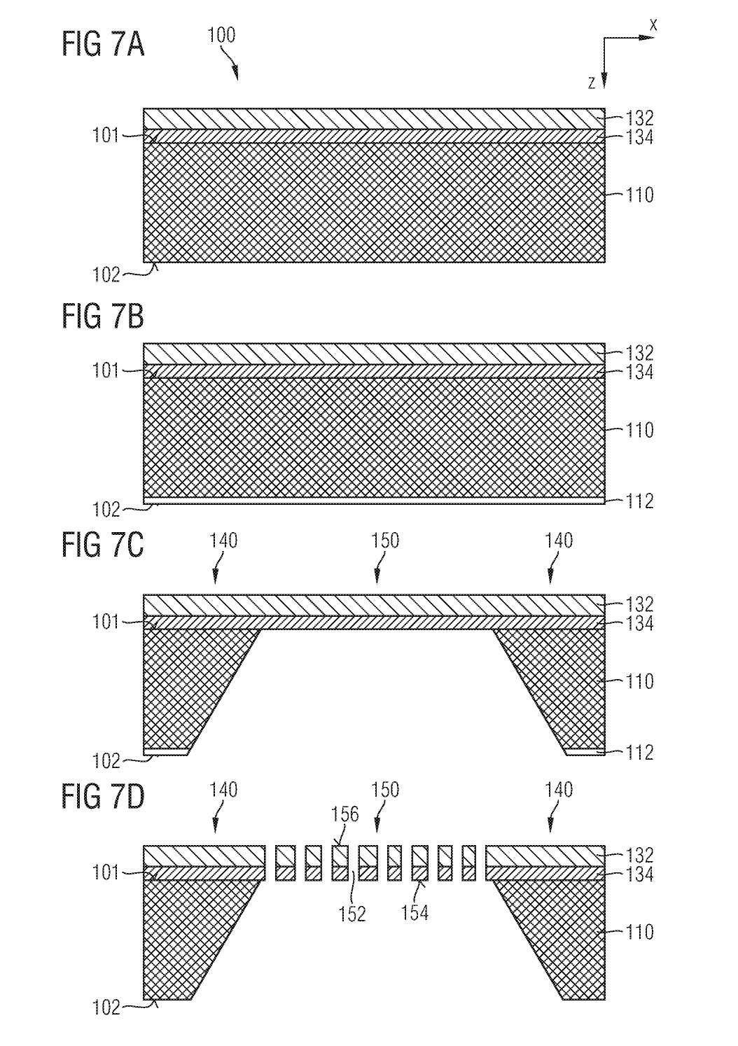

FIG. 6 is a cross-sectional view of a microfiltration device 100 according to another embodiment. As can be seen from comparing FIG. 5G and FIG. 6, the microfiltration device 100 of FIG. 6 comprises the same structure as the microfiltration device 100 of FIG. 5G, subject to providing a porous semiconductor layer constituting the first layer 132 within the filter part 150. The microfiltration device 100 of FIG. 6 is manufactured in a comparable way as the microfiltration device of FIG. 5G, subject to the process step shown in FIG. 5E. Instead of performing an lithographical patterning of the second layer 134 followed by an electro-chemical wet-etching step, the second layer 134 is removed in the complete filter part 150 of the first layer 132, followed by an process to generate porous semiconductor material within the filter part 150. The silicon is made porous using an anodisation process involving electrochemical wet etching, for example.

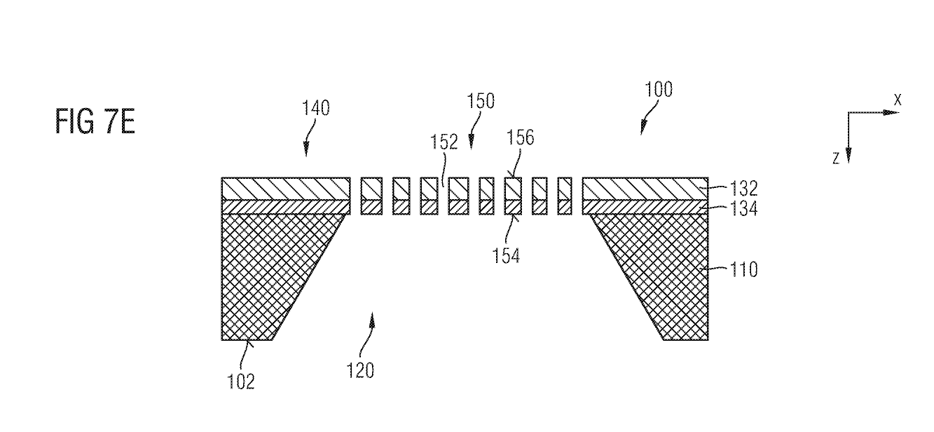

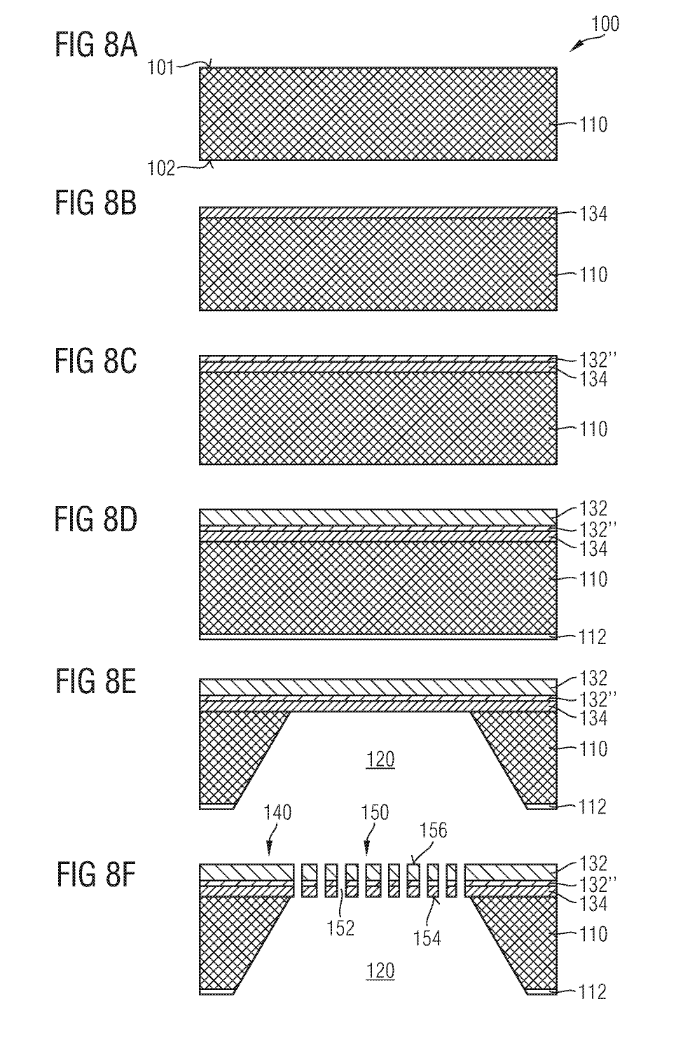

FIG. 7E is a schematic cross-sectional view of a portion of a microfiltration device 100 of another embodiment.

As can be seen from FIG. 7E, the microfiltration device 100 may also be formed by using an semiconductor-on-insulator (SOI)-manufacturing process. Herein, the second layer 134 of an insulating material is formed on the substrate 110 comprising a semiconductor material. For example, the second layer 134 may be a silicon oxide layer formed on the substrate 110 of silicon. On the second layer 134, the first layer 132 is formed. The first layer 132 comprises a semiconductor material such as silicon. Thus, the microfilter 130 includes a double layer structure of the second layer 134 and the first layer 132, wherein the second layer 134 is in contact with the substrate 110 in the frame part 140.

FIG. 7A to FIG. 7D are cross-sectional views illustrating a method of manufacturing a microfiltration device 100 of FIG. 7E.

As can be seen from FIGS. 7A and 7B, a substrate 110 of a semiconductor material is provided, on which an second layer 134 of an insulating material is formed. The semiconductor material may be silicon, the insulating material may be silicon oxide. On the second layer 134, the first layer 132 of a semiconductor material such as silicon is formed, e.g. by epitaxial growth or deposition. Thereafter, a masking layer 112 is formed on the second surface 102 of the substrate 110. The masking layer 112 may comprise an insulating material such as silicon oxide.

As shown in FIG. 7C, the masking layer 112 is patterned such that the second surface 102 of the substrate 110 is freely exposed at a area of overlapping the filter part 150 of the microfilter 130. Thereafter, the cavity 120 is etched by a wet-chemical etching process.

As shown in FIG. 7D, in a further step, the second layer 134 is patterned and used as a etching mask layer to form pores 152 in the filter part 150 being extended through the second layer 134 and the first layer 132 by an anisotropic plasma etching process, for example. When using an anisotropic plasma etching process, critical pore dimensions may be maintained.

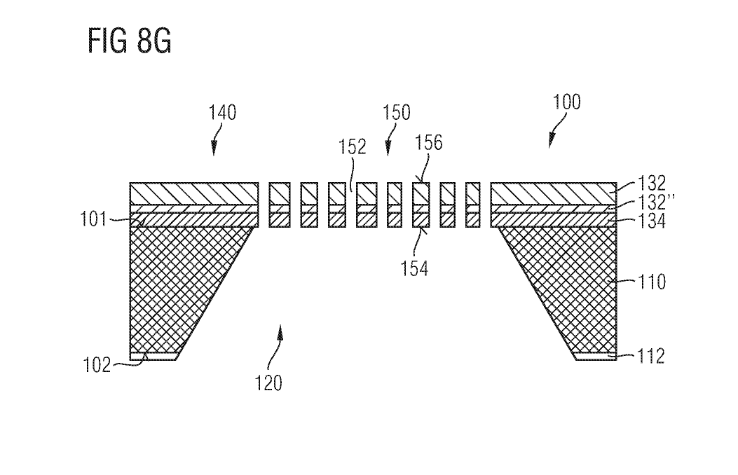

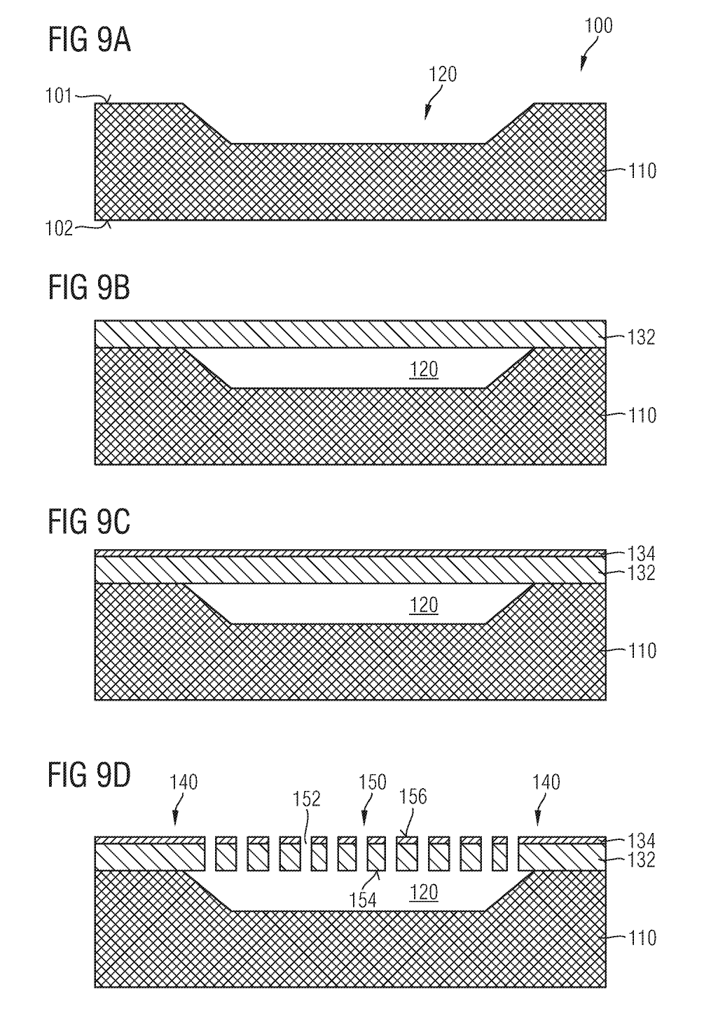

FIG. 8G shows a schematic cross-sectional view of a portion of a microfiltration device 100 according to another embodiment.

As can be seen from comparing the microfiltration device 100 of FIG. 7E with the microfiltration device of FIG. 8G, the microfiltration device 100 of FIG. 8G has a structure comparable to the microfiltration device 100 of FIG. 7E, subject to providing a polysilicon layer 132″ on the second layer 134. The polysilicon layer 132″ is a layer of polysilicon deposited on the second layer 134, wherein the first layer 132 of polysilicon is formed by epitaxial growth. This is a relatively low cost manufacturing process in comparison to that used for the microfiltration device of FIG. 7E, where the SOI substrate costs are relatively high.

FIG. 8A to 8F are cross-sectional views illustrating a method of manufacturing a microfiltration device 100 of FIG. 8G.

Since the method of manufacturing the microfiltration device 100 of FIG. 8G is basically the same as the manufacturing method for the microfiltration device 100 of FIG. 7E, only the differences in the manufacturing process will be explained hereinafter. As can be seen from FIGS. 8A and 8B, the substrate 110 is provided, on which the second layer 134 of an insulating material is formed. As can be seen from FIGS. 8C and 8D, the polysilicon layer 132″ is deposited on the second layer 134 comprising silicon oxide. On the deposited polysilicon layer 132″, the first layer 132 of polysilicon is formed on the polysilicon layer 132″ by epitaxial growth. The manufacturing steps in FIGS. 8E and 8F are comparable to the manufacturing steps in FIGS. 7C and 7D and will not be further explained in detail.

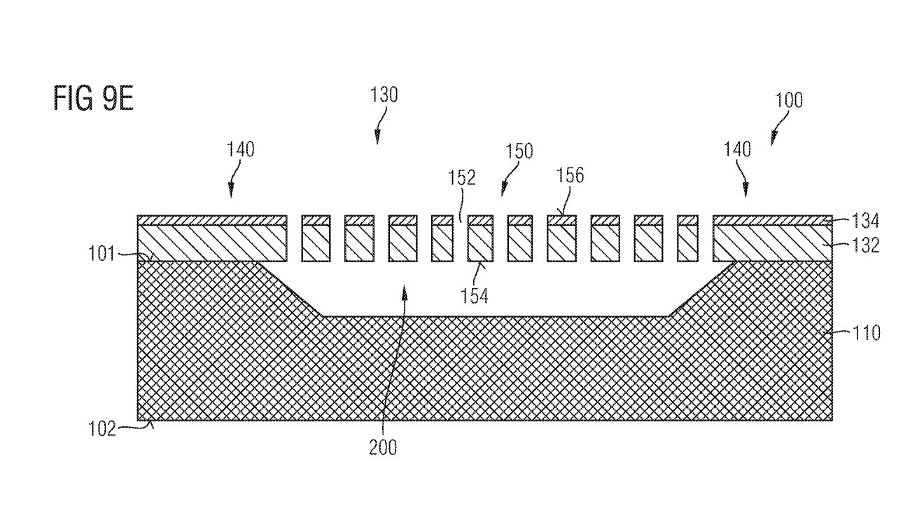

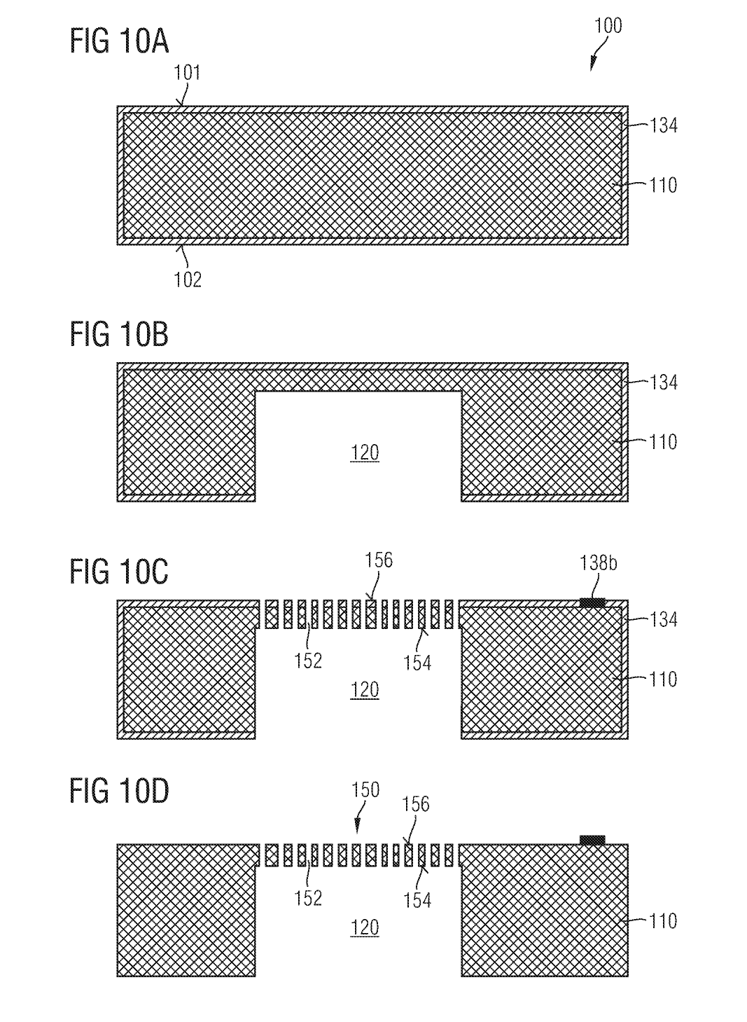

FIG. 9E is a schematic cross-sectional view of a portion of a microfiltration device 100 according to another embodiment.

According to the embodiment of FIG. 9E, the microfilter 130 is bonded on the substrate 110. The microfilter 130 comprises the first layer 132 of a semiconductor material such as silicon and the second layer 134 of an insulating material such as silicon oxide. The substrate 110 may include an insulating material such as glass or undoped silicon, for example.

FIG. 9A to 9D are cross-sectional views illustrating a method of manufacturing a microfiltration device of FIG. 9E.

In a first step (FIG. 9A), a substrate 110 is provided, in which a cavity 120 is formed by lithographically patterning an etching mask layer, followed by an etching process. According to another embodiment, the substrate 110 may comprise polydimethylsiloxane (PDMS), wherein the cavity 120 is formed within the substrate 110 by a stamp process. Alternatively, the substrate 110 may also comprise a semiconductor material such as silicon, for example.

As shown in FIG. 9B, the microfilter 130 comprising the first layer 132 of a semiconducting or conducting material is bonded to the substrate 110 such that the frame part 140 comes into contact with the substrate 110 and the filter part 150 abuts the cavity 120.

As shown in FIG. 9C, the second layer 134 of an insulating material is formed on the first layer 132.

As shown in FIG. 9D, the second layer 134 of an insulating material such as an oxide is patterned by a lithographical process and used as an etching mask in an etching process to form the pores 152 within the filter part 150.

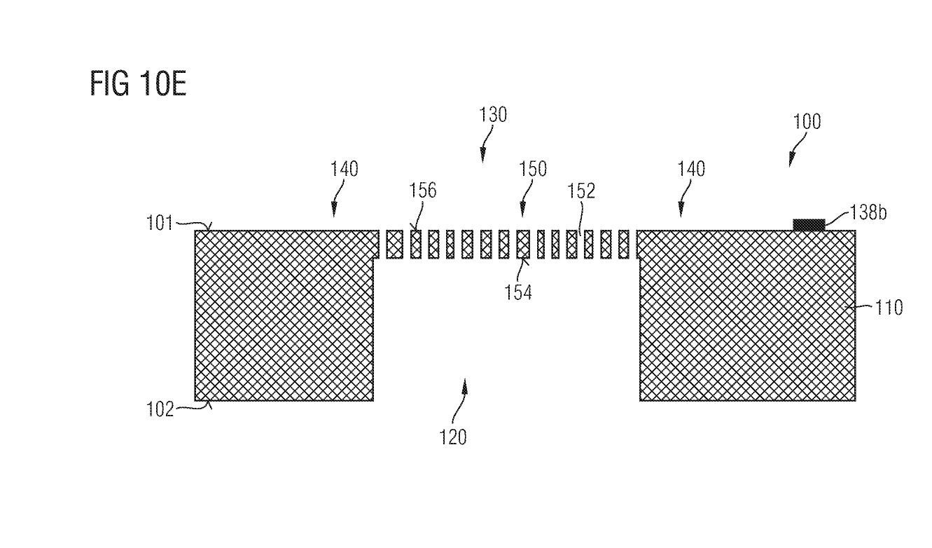

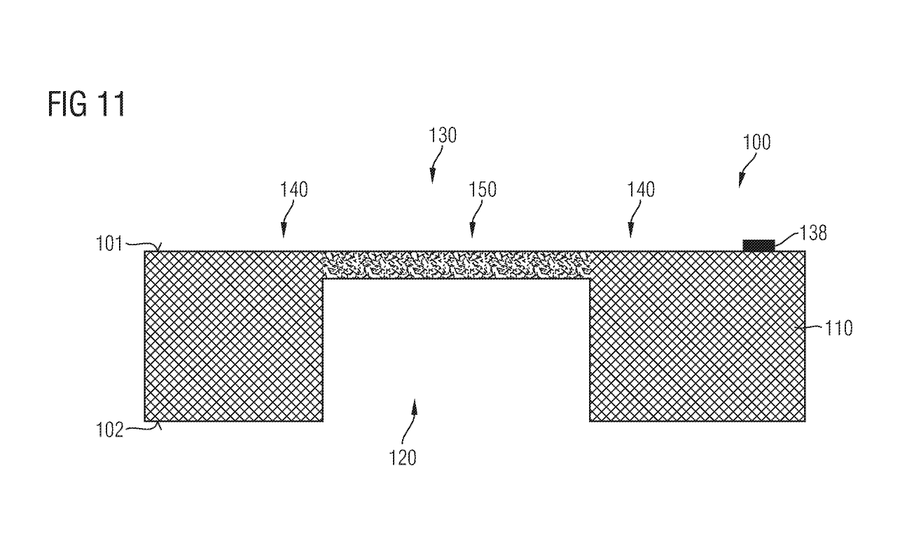

FIG. 10E is a cross-sectional view of a portion of a microfiltration device 100 according to another embodiment.

As shown in FIG. 10E, the microfilter 130 and the substrate 110 comprise a same material. Thus, the substrate 110 comprises a semiconducting or conducting material. The microfilter 130 may be electrically contacted by means of the contact pad 138b of the contact area 138.

FIG. 10A to 10D are cross-sectional views illustrating a method of manufacturing a microfiltration device 100 of FIG. 10.

As shown in FIG. 10A, the substrate 110 of a semiconducting or conducting material, such as silicon, is provided, wherein the second layer 134 of an insulating material is formed on the first surface 101 and the second surface 102 of the substrate 110.

As shown in FIG. 10B, the second layer 134 is patterned and used as a etching mask layer to form the cavity 120 by deep reactive ion etching (DRIE) process. The deep reactive ion etching (DRIE) process may include an anisotropic plasma etching process using an appropriate etch gas, e.g. at least one of SF₆, Cl₂, Br₂, CCl₄, CHCl₃, CHBr₃, BCl₃, and HBR.

As shown in FIG. 100, the second layer 134 is then patterned at the first surface 101 and used as a etching mask to form the pores 152 within the filter part 150 by an etching process.

After removing the second layer 134 from the surface of the substrate 110 (FIG. 10D), the microfiltration device 100 of FIG. 10E is provided.

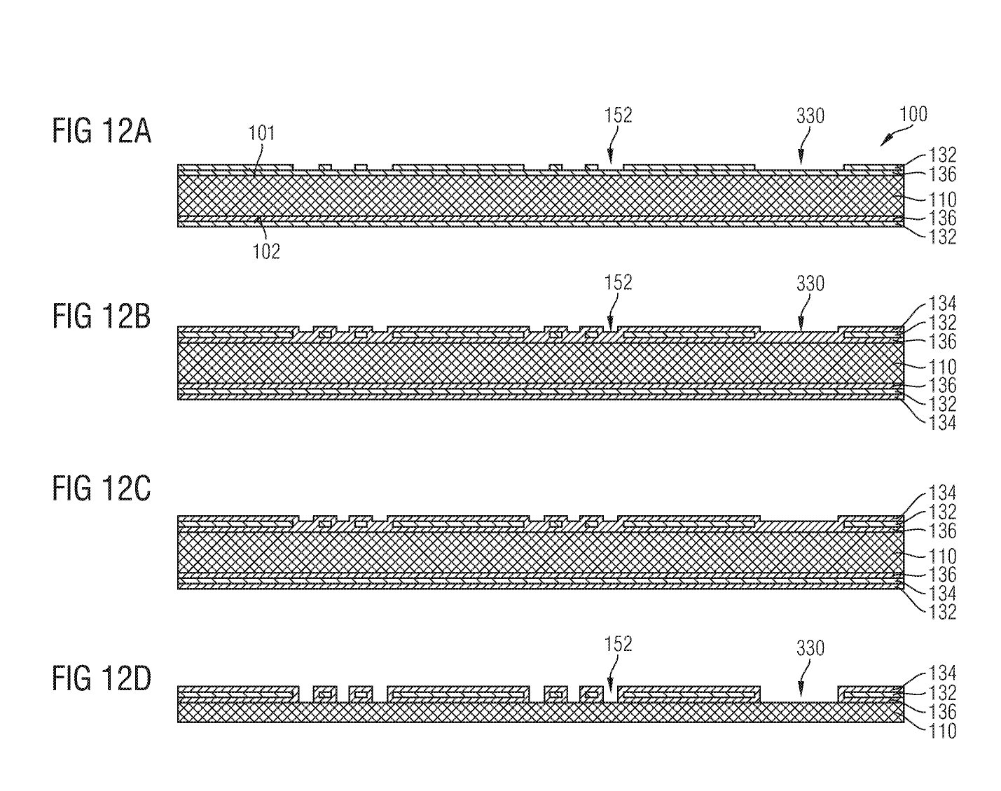

FIG. 11 shows a cross-sectional view of a microfiltration device 100 according to another embodiment.

As can be seen from comparing FIGS. 10E and 11, the microfiltration device 100 of FIG. 11 differs from the microfiltration device 100 of FIG. 10E in that the filter part 150 does not comprise a plurality of pores 152 being extended from the first surface 101 to the cavity 120, but comprises a porous semiconductor material such as porous silicon in the filter part 150. The method for manufacturing the microfiltration device of FIG. 11 is comparable to the manufacturing method as shown in FIG. 10A to FIG. 10D subject to removing the second layer 134 in the complete area of the filter part 150, followed by an etching process for generating porous silicon in the filter part 150.

In the embodiments of FIG. 3H to FIG. 11, microfiltration devices 100 have been described, which may be formed of single-crystalline silicon or epitaxial poly-silicon which allows filtration of blood components, for example, including the blood-plasma based on lithographically defined and etched pores 152. Microfiltration devices 100 using silicon or epitaxial poly-silicon are elastic and robust compared to silicon nitride and polymer membranes. Beside, the silicon membrane is not too thick and can be manufactured in a semiconductor fab. The microfiltration devices 100 as described above, in particular the electro-chemically wet-etched silicon microfiltration devices of FIGS. 3H and 4, the electro-chemically double-side wet-etched silicon microfiltration device 100 of FIG. 5G, the electro-chemical double-side wet-etched silicon microfiltration device 100 having two cavities in the same substrate and porous arrangement of FIG. 6, the anisotropic plasma etched silicon-on-insulator (SOI) based microfiltration device 100 of FIG. 7E, the wet-chemically etched epi-polysilicon-on-insulator based microfiltration device of FIG. 8G, the bonded and etched silicon microfiltration device 100 of FIG. 9E, the deep reactive ion etched (DRIE) silicon microfiltration device 100 according to FIG. 10E or the deep reactive ion etched (DRIE) silicon microfiltration device 100 with porous arrangement according to FIG. 11 are embodiments of a microfiltration device 100, wherein certain features or structures of the different embodiments can be combined, if not explicitly mentioned otherwise. For example, the filter parts 150 of the embodiments as shown in FIGS. 3H and 4 may also comprise a porous semiconductor layer instead of pores 152, as shown in the embodiment of FIG. 6.

In the following, two further embodiments of a microfiltration device 100 will be described.

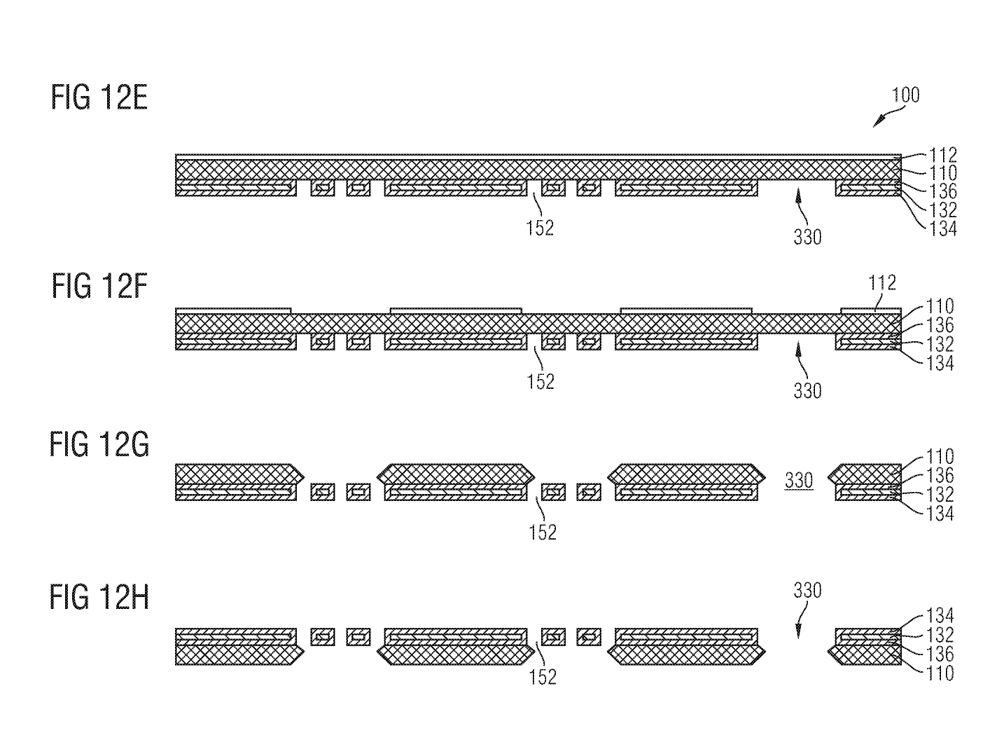

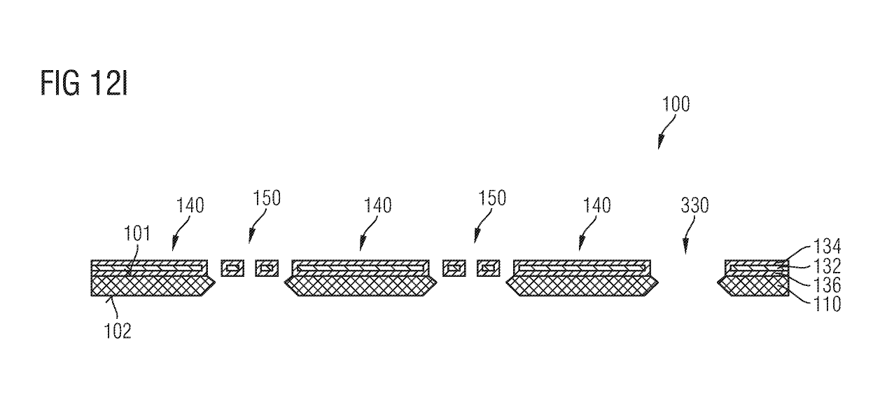

FIG. 12I is a schematic cross-sectional view of a portion of a microfiltration device 100 according to another embodiment.

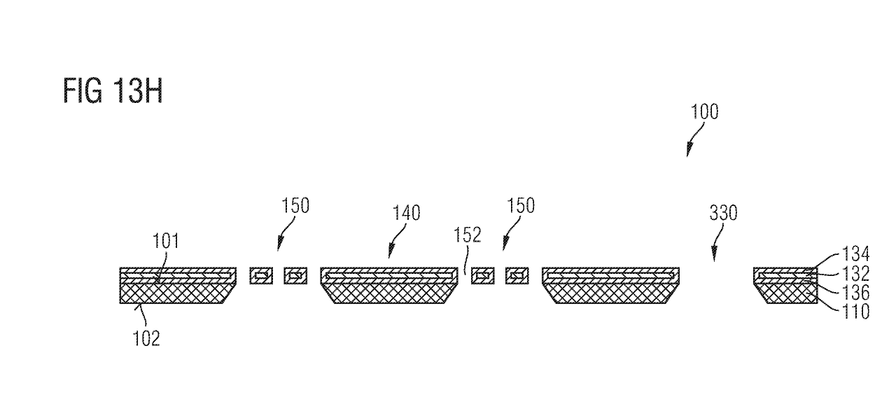

As can be seen from FIG. 12I, the microfiltration device 100 comprises the first layer 132 of the semiconducting or conducting material, the second layer 134 of an insulating material, and further comprises a third layer 136 of an insulating material, wherein the first layer 132 is sandwiched between the second layer 134 and the third layer 136. Thus, the microfiltration device 100 comprises a substrate 110, on which the third layer 136 of an insulating material is formed. On the third layer 136, the first layer 132 of a semiconducting or conducting material is formed, and on the first layer 132, the second layer 134 of an insulating material is formed. As can be seen from FIG. 12I, the microfiltration device 100 comprises a microfilter 130 including a frame part 140 in contact with the substrate 110, a filter part 150 abutting the cavity 120, and further comprises a venting hole part 330, which will be discussed in all detail with regard to FIG. 14.

FIG. 12A to 12H are cross-sectional views illustrating a method of manufacturing a microfiltration device 100 of FIG. 12I.

As shown in FIG. 12A, the substrate 110 is provided, on which on both on the first surface 101 and the second surface 102, the third layer 136 and the first layer 132 are formed. On the side of the first surface 101 of the substrate 110, the first layer 132 is lithographically patterned to form the pores 152 and the venting hole 330 being extended through the first layer 132 only.

As shown in FIG. 12B, the second layer 134 is formed on both sides of the substrate 110 such that the second layer covers the patterned first layer 132 and the third layer 136 on the side of the first surface 101, and further covers the third layer 136 being not patterned and the first layer 132 on the side of the second surface 102 of the substrate 110. The substrate 110 may comprise silicon, the third layer 136 and the second layer 134 may comprise a chemical vapor deposition layer comprising silicon nitride or silicon carbide, and the first layer 132 may comprise polysilicon.

As shown in FIGS. 12C and 12D, the pores 152 and the venting hole 330 are formed to be extended through the second layer 134, the first layer 132 and the third layer 136 by means of a lithographical process.

As can be seen from FIG. 12D, the side of the second surface 102 of the substrate 110 is treated such that the layer structure of the first to third layers 132, 134 and 136 are removed together with a part of the substrate 110 abutting the second surface 102. The removal of these layers and the part of the substrate 110 may be performed by a substrate thinning process such as a mechanical grinding and polishing, or a wet chemical etching, or a chemical mechanical polishing (CMP) process.

As shown in FIG. 12E, a masking layer 112, e.g. a chemical vapor deposition layer comprising an oxide, undoped silicate glass (USG) or carbon is deposited on the second surface 102 formed by the substrate thinning process. After patterning the masking layer 112 to expose areas overlapping with the filter parts 150 and the venting hole 330, an electro-chemical wet-etching process is performed to form the cavity 120 abutting the filter part 150 of the microfilter 130 and to form a through hole through the microfiltration device 100 in the area of the venting hole 330 (FIG. 12G).

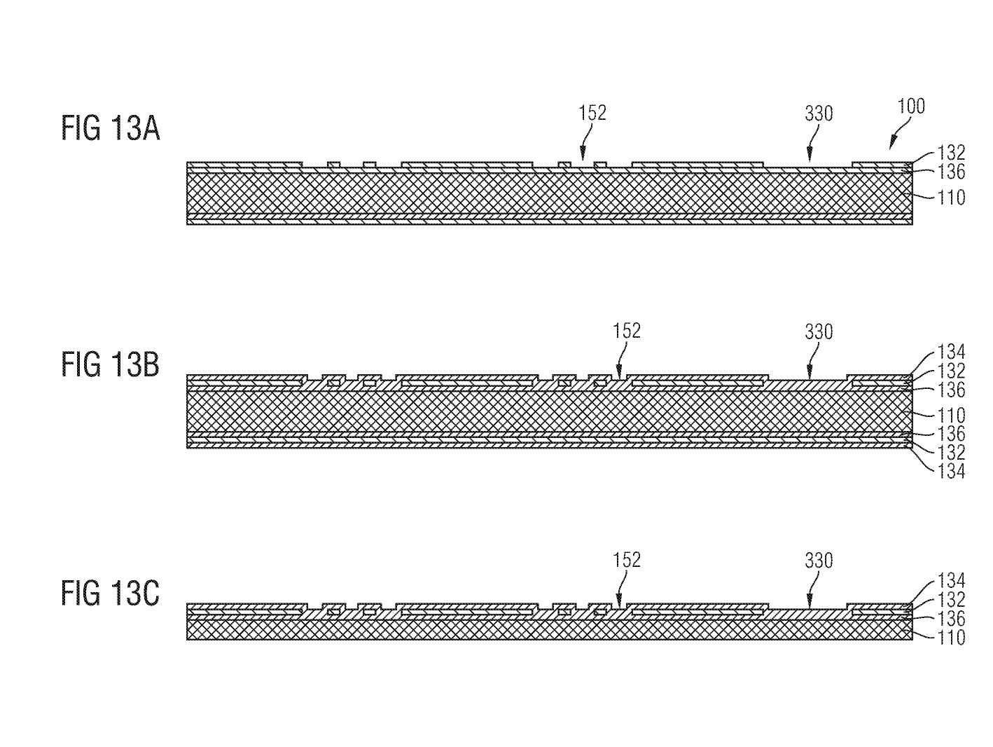

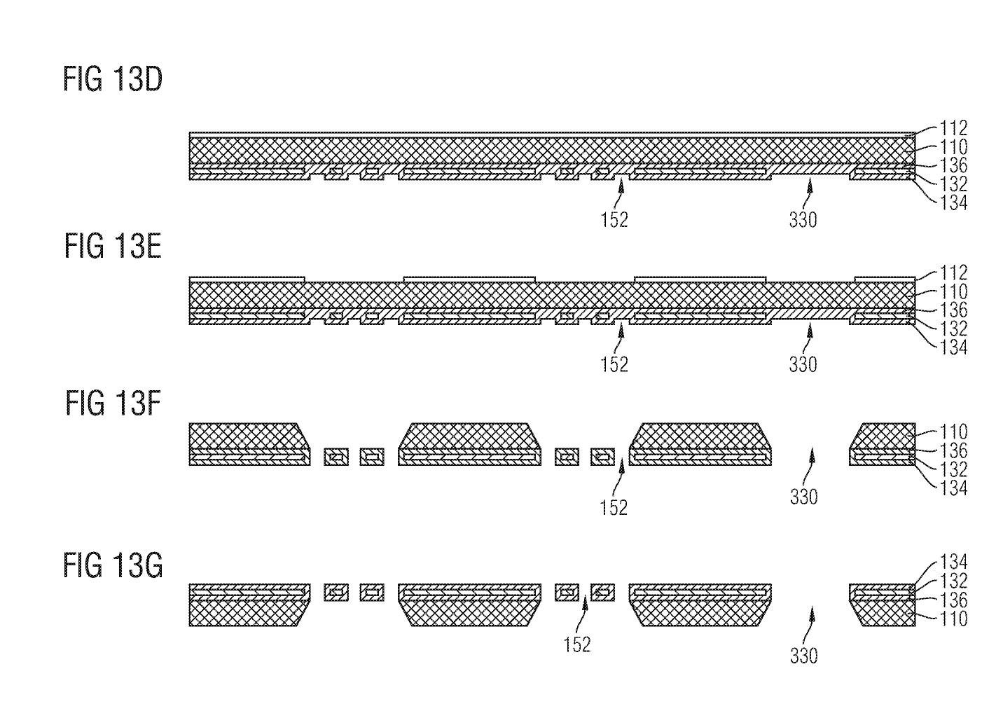

By flipping the microfiltration device 100 in a vertical direction, the microfiltration device 100 of FIG. 13H is provided.

FIG. 13H is a schematic cross-sectional view of a portion of a microfiltration device 100 according to another embodiment.

As can be seen from a comparison of FIG. 12I and FIG. 13H, the microfiltration device 100 of FIG. 13H differs from the microfiltration device 100 of FIG. 12I only in the etching profile of the substrate 110. The difference in the structure of the cavity 120 can be best understood from the manufacturing process, which is shown in FIG. 13A to 13G.

As can be seen from FIGS. 13A and 13B, the process of manufacturing the microfiltration device 100 of FIG. 13H is the same as already shown with regard to FIGS. 12A and 12B.

As shown in FIG. 13C, however, no lithographical step is performed before the substrate thinning process, e.g. a mechanical grinding and polishing, or a wet chemical etching, or a chemical mechanical polishing (CMP) process.

The process steps in FIGS. 13D and 13E are again comparable to the process steps as shown in FIGS. 12E and 12F.

As can be seen from FIG. 13F, the cavity 120 and the pores 152 as well as the venting hole 330 are formed by a plasma etching process performed from the side of the second surface 102 only. After flipping the microfiltration device 100 in a vertical direction (FIG. 13G), the microfiltration device 100 of FIG. 13H is provided.

As can be seen from FIGS. 12I and 13H, a microfiltration device 100 may use a thin-film membrane stack consisting of a dielectric (oxide or nitride or carbide), a conductive layer (polysilicon) which has stiffness tunability and a third dielectric layer (oxide or nitride or carbide), which are elastic and robust compared to mono-layer silicon nitride membrane or polymer membrane. Micro-size pores 152 may be etched into the membrane stack using lithographically defined and etched pores 152. Besides, the triple-stack membrane of the third layer 136, the first layer 132 and the second layer 134 is not too thick and can be manufactured in a semiconductor fab.

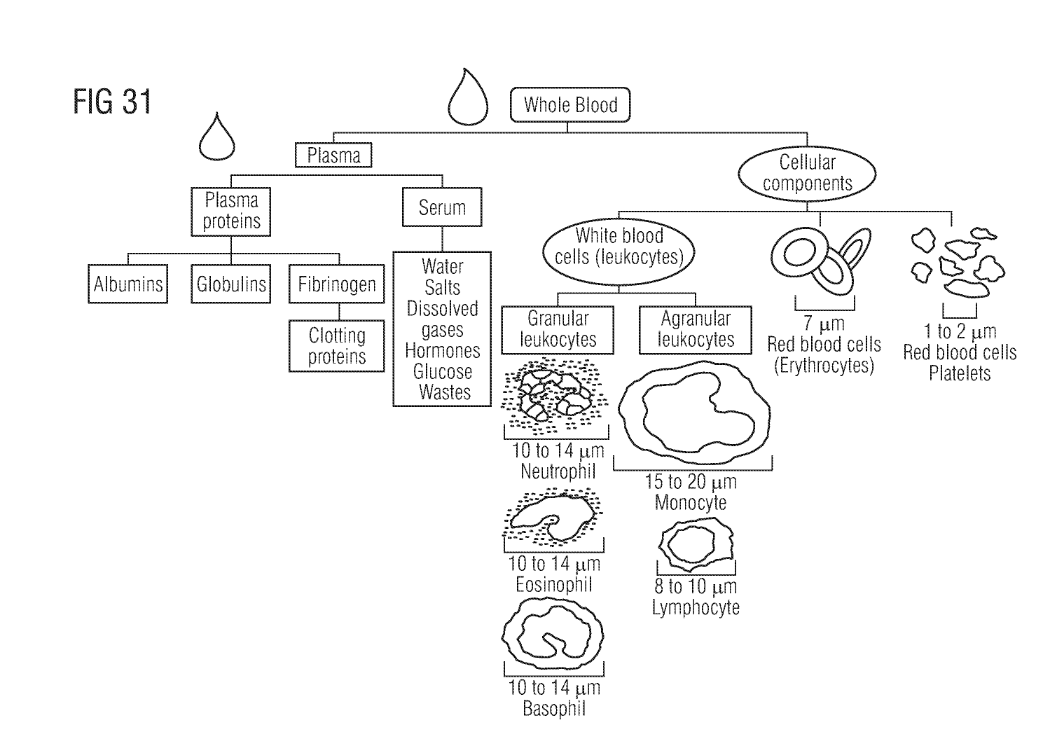

As can be seen from FIG. 3H to FIG. 13H, the microfiltration device 100 comprises a microfilter 130 including a frame part 140 in contact with the substrate 110 and a filter part 150 abutting the cavity 120, wherein the microfilter 130 comprises in both the frame part 140 and the filter part 150 a semiconducting or conducting material. To achieve a filtration effect of the filter part 150, the filter part 150 may comprise porous semiconductor such as porous silicon or may comprise pores 152 extended from the side of the first surface 101 of the substrate to the cavity 120. The filter part 150 may comprise pores 152 arranged in a regular pattern. The filter part 150 may also comprise pores 152 of a uniform pore size, wherein the uniform pore size may be a size in a range of 1 μm to 10 μm, and depends on the application. According to an embodiment, the microfiltration device 100 may be adapted to separate a blood probe into a plasma component and cellular components as shown in FIG. 31. Thus, according to an embodiment, the filter part 150 of the microfilter 130 may constitute a microsieve having a plurality of pores 152 arranged in a regular pattern and having a uniform pore size. Uniform pore size means the provision of a plurality of pores 152 having a narrow pore size distribution. For example, the pore size distribution, i.e. the standard deviation may be less than 5%, or less than 2%, or less than 1%. However, it is also possible to provide a plurality of pores 152 in the filter part 150 having different pore sizes, wherein the pores 152 with the biggest pore size define the maximum size of the filtrate components. The mean distance between the pores 152 may be in a range of 2 μm to 10 μm, depending on the required robustness. The ratio of opening area of the pores 152 and the complete area of the filter part 150 may be in a range between 0.1 to 0.7.

FIG. 14 is a schematic cross-sectional view of a portion of a sensor device according to an embodiment.

As can be seen from FIG. 14, the microfiltration device 100 of FIGS. 12I and 13H having a stacked layer of the third layer 136, the first layer 132 and the second layer 134 may be employed in a sensor device 300, in which a sensor 200 is located at a filtrate side 154 of the filter part 150. The sensor 200 is adapted to measure a characteristic of a filtrate 620, which is filtered by the filter part 150 from a feed 610 from a fluid such as blood. The sensor device 300 comprises a reservoir 310 to receive a feed 610. As can be seen from FIG. 14, the cavity 120 may be on a feed side 156 or on a filtrate side 154, depending on the mounting of the microfiltration device 100 within the sensor device 300. The feed 610 may be a fluid of medical interest such as blood of a human or an animal. However, the sensor device 300 may be employed also in further fields of biological and chemical analytics. Thus, the microfiltration device 100 may be employed, together with the sensor device 300 in the field of gas sensoric, wherein the microfiltration device 100 constitutes a gas filter or a protection for the sensor 200 from dust particles of a certain size. Furthermore, the sensor device 300 may be employed in analyzing water quality.

As can be seen from FIG. 14, the microfiltration device 100 is bonded to a sensor substrate 210 on which the sensor 200 is formed by means of a bonding 320. The bonding 320 may constitute a spacer to form a measurement chamber 325 between the sensor substrate 210 and the microfiltration device 100. The bonding 320 further seals the gap between the sensor substrate 210 and the microfiltration device 100 to form the measurement chamber 325. The feed 610 is introduced from the reservoir 310 into the measurement chamber 325 through the filter part 150 of the microfilter 130 of the microfiltration device 100 as a filtrate 620 after filtration by the filter part 150. According to an embodiment, the vent-hole 330 is provided to facilitate the introduction of filtrate 620 into the measurement chamber 325. The sensor 200 may comprise a sensor electrode 220 as shown in FIG. 14. However, the sensor 200 may comprise different kind of sensors, as will be discussed below. The sensor electrode 220 may be formed by depositing or sputtering or implantation. The first layer 132 in the microfilter 130 may constitute a further sensor electrode of the sensor 200.

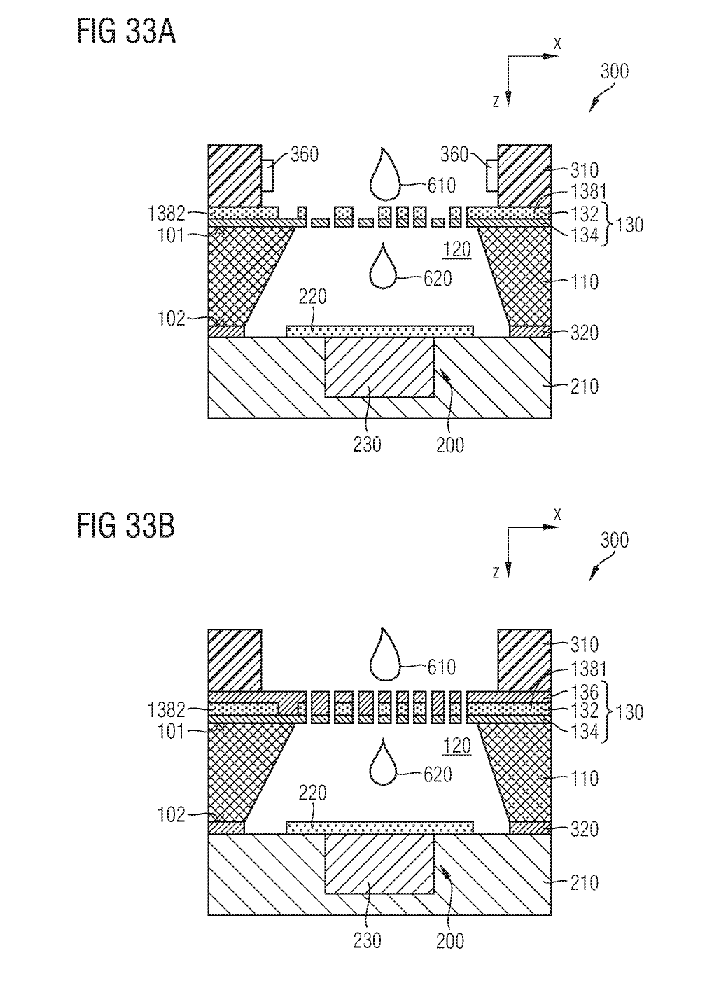

Thus, by applying a voltage between the contact area 138 being electrically connected to the semiconducting or conducting material in the filter part 150 and the sensor electrode 220 formed on the sensor substrate 210, the sensor electrode of the first layer 132 and the sensor electrode 220 on the sensor substrate 210 form an impedance (capacitive) or an amperometric sensor. Additionally, the electrodes of the first layer 132 and the sensor electrode 220 can be configured into an electrospray for electrostatic fluid delivery induced by dripping for delivery of blood-plasma to the sensor electrode 220, to avoid a clogging of the pores 152 and to minimize the filtration time in comparison to differential-pressure based fluid delivery. An analytical estimation for a single pore 152 is shown in FIG. 33. As can be seen from FIG. 33, based on the gap between the electrode of the first layer 132 and the sensor electrode 220, the required applied voltage for micro-dripping mode for human blood is illustrated.

Thus, as shown in FIG. 14, a microfiltration device 100 made of multilayered (triple-stacked) thin-film membrane stack having predefined pores 152 allowing for filtration of blood components including the blood-plasma may be employed in a sensor device 300, wherein the micro-size pores 152 are lithographical defined and etched into the multi-layered membrane stack. The triple-stack of the third layer 136, the first layer 132 and the second layer 134 comprises a conductive middle layer, covered by dielectric top and bottom layers. The middle layer is formed with polysilicon whose stiffness can be tuned by implantation and moreover this layer can also be made electrically active. When this triple-stack MEMS filter chip, i.e. the microfiltration device 100 is combined with a sensor substrate 210 having a sensor electrode 220, the electrodes constituted by the first layer 132 and the sensor electrode 220 can be used for impedance (capacitive) or amperometric sensing. Additionally, a filtrate such as a blood-plasma can be collected by applying a potential difference between the electrode of the first layer 132 and the sensor electrode 220 for electrostatic fluid delivery using the dripping mode of an electrospray, to avoid clogging and minimize the filtration time. Herein, the filter part 150 constitutes the sensor electrode of the sensor 200.

FIGS. 15 and 16 show schematic cross-sectional views of different embodiments of a sensor device 300. The microfilter 130 is shown as a single layer structure. However, all microfiltration devices 100 as discussed above with regard to FIGS. 3H to 13H may be applied in the sensor devices 300 as discussed with regard to FIGS. 14 to 30, 33A, 33B, 34A and 34B.

According to an embodiment, the sensor device 300 works as a bio-sensor chip comprising the microfiltration device 100 and the sensor 200. The sensor device 300 allows the detection and measurement of various blood-related parameters for point-of-care testing (POCT). By means of the sensor device 300, patients are enabled to measure various blood-related parameters at their homes, conveniently, using the sensor device 300 as a point-of-care testing device. Compared to several other medical diagnostic solutions, the plasma of blood is first filtered out of the blood and then the filtrate is measured for various body condition related parameters.

As can be seen from FIGS. 15 and 16, the sensor 200 comprises the sensor electrode 220. The sensor 200 may further comprise an application-specific integrated circuit (ASIC) 230. By means of the sensor electrode 220 and the read-out application-specific integrated circuit 230 in the sensor substrate 210, the sensor 200 is able to detect at least one blood-parameter related to the body condition of patients, like glucose level, infection, hormones, salts, for example.

As can be seen from comparing FIGS. 15 and 16, the cavity 120 may be on a filtrate side (FIG. 15) or on a feed side (FIG. 16). The sensor device 300 as shown in FIGS. 15 and 16 may have an electrical read-out. The microfiltration device 100 may also be applied on top of a gas-chemical sensor 200 for protection from dust and particles. Furthermore, the first layer 132 in the microfilter 130 acting as an additional electrode may be applied to sense electrical properties of components of the feed 610 which have not passed the filter part 150.

FIGS. 17 and 18 show further embodiments of a sensor device 300. In the embodiments of FIGS. 17 and 18, the sensor 200 may comprise an optical sensor 240. The optical sensor 240 may be connected with the application-specified integrated circuit 230 to provide an opto-electronic read-out. The sensor devices 300 as shown in FIGS. 17 and 18 may also be employed as a gas-chemical sensor device, wherein the microfiltration device 100 protects the sensor 200 from dust and particles. As can be seen from FIGS. 17 and 18, the difference between the sensor devices 300 is again the location of the cavity 120, wherein the cavity 120 is either arranged on the filtrate side 154 (FIG. 17) or on the feed side 156 (FIG. 18).

As can be seen from FIGS. 14 to 18, the sensor 200 is arranged on a sensor substrate 210, wherein the sensor substrate 210 is bonded to the microfiltration device 100 such that the sensor 200 faces the filter part 150. By this arrangement of the sensor 200 with respect to the filter part 150, an assembling of the sensor device comprising the sensor 200 and the microfiltration device 100 may be facilitated. However, it is also possible that the sensor 200 is arranged such that it does not face the filter part 150.

FIG. 19 is a schematic cross-sectional view of a sensor device 300 according to another embodiment.

As can be seen from FIG. 19, the microfilter 130 includes the frame part 140 in contact with the sensor substrate 210 and a filter part 150 abutting the cavity 120. The filter part 150 is, according to the embodiment of FIG. 19, a surf ace-micromachined membrane manufactured by processes used for micro-mechanical systems (MEMS). The microfilter 130 comprises in both the frame part 140 and the filter part 150 a semiconducting or conducting material. According to an embodiment, the frame part 140 and the filter part 150 comprise a semiconductor material such as silicon.

According to the embodiment of FIG. 19, the sensor electrode 220 is arranged on the sensor substrate 210. However, as can be seen from the embodiment of FIG. 2C, the sensor electrode 220 of the sensor 200 may also be arranged such that it is part of the filter part 150. As already discussed above, the first layer 132 of a semiconducting or conducting material may constitute the sensor electrode 220. However, it is also possible that a separate metallization layer is provided at a filtrate side 154 of the filter part 150.

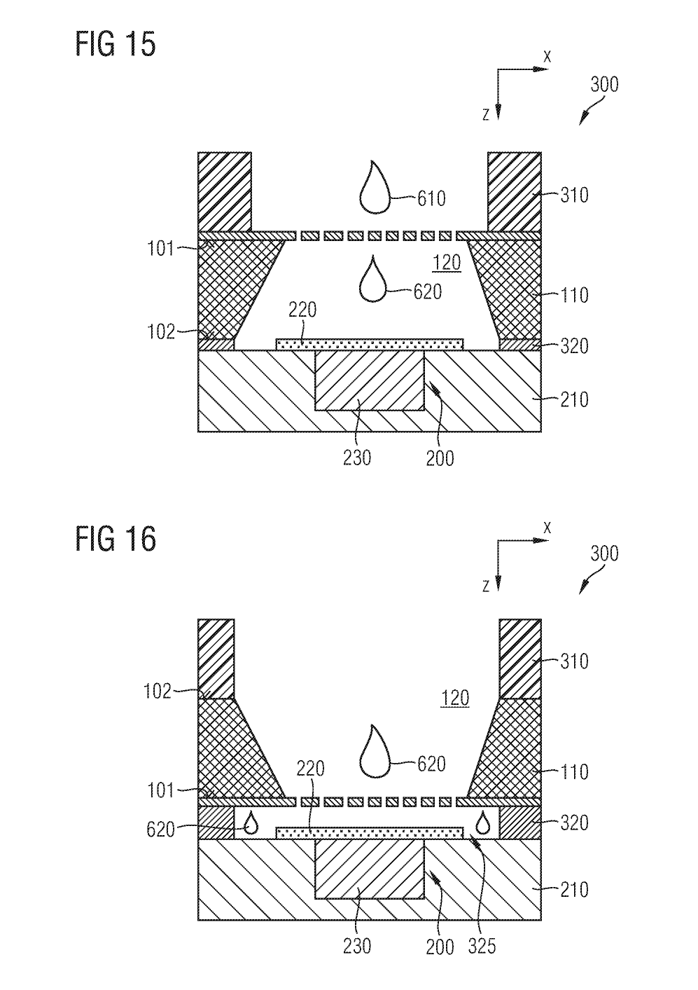

FIG. 21 shows a schematic perspective view of a filter part 150 of a surface-micromachined MEMS microfilter, wherein FIG. 22 is a schematic cross-sectional view along a section plane A-A′ of FIG. 21.

As can be seen from FIG. 22, the filter part 150 has a length in a range between 10 μm and 1000 μm. The layer thickness H1 may be in a range between 100 nm and 5 μm. The cavity height H2 may be in a range between 100 nm and 20 μm.

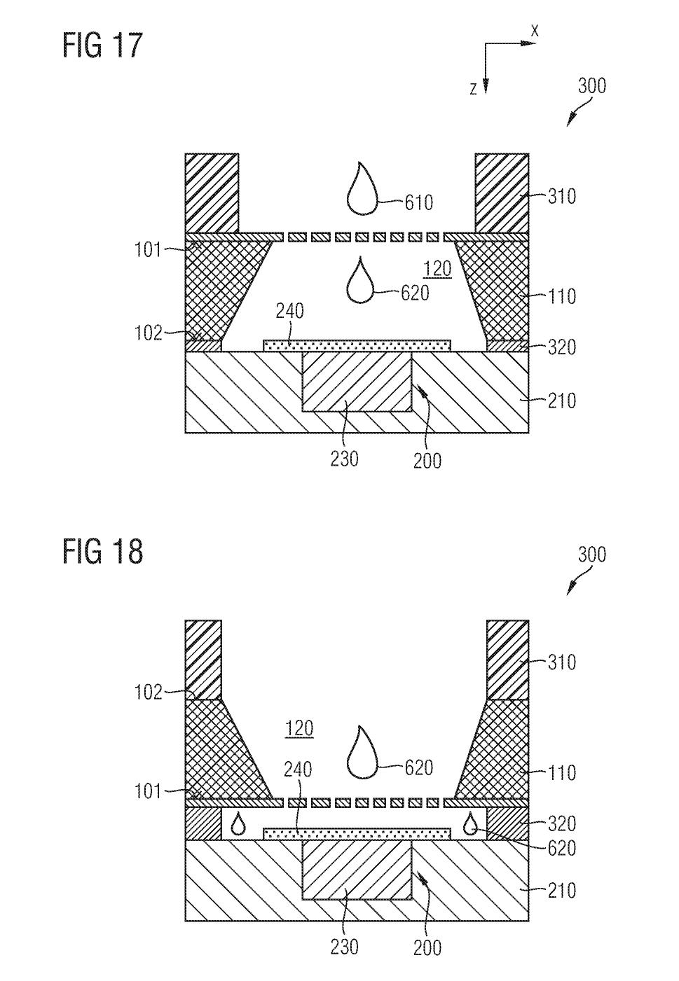

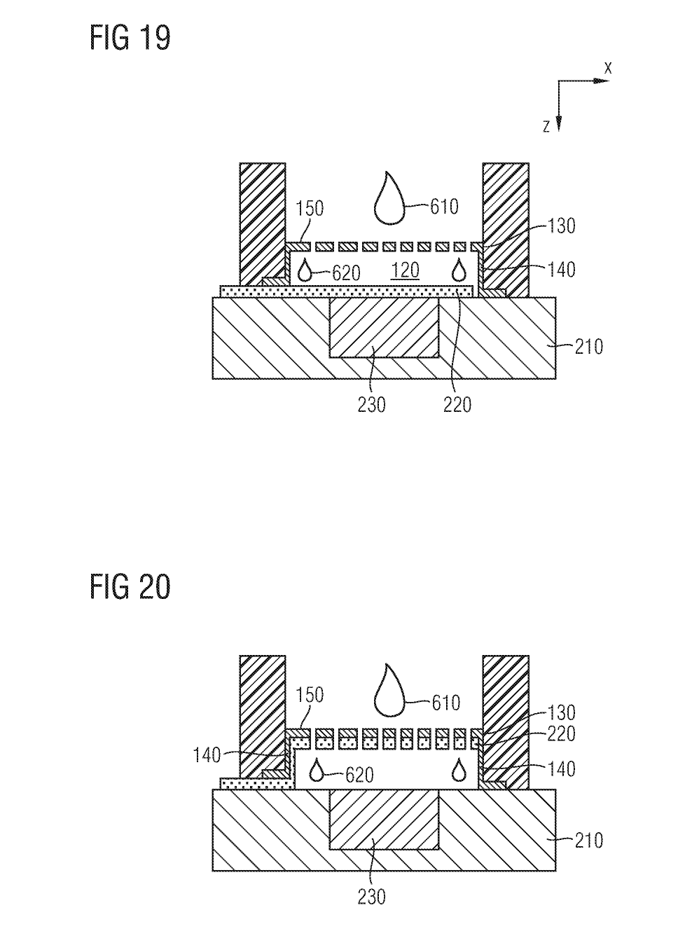

According to an embodiment of the microfilter 130 of FIG. 21, supporting pillars 340 may be employed to support the filter part 150 on the sensor substrate 210. The supporting pillars 340 are arranged on the first surface 101 of the sensor substrate 210 and abut the filtrate side 154 of the filter part 150. The supporting pillars 340 are shown in FIG. 23 and FIG. 24. Due to the supporting pillars 340, the semiconductor membrane 350 shows an improved robustness and stability against mechanical loads. The maximal unsupported side-length LS may be in a range between 5 μm to 100 μm. The ratio between the layer thickness H1 of the filter part 150 and the maximum unsupported side-length LS may depend on the placement of the supporting pillars 340. The supporting pillars 340 may be arranged in a regular pattern as shown in FIG. 24. For example, the supporting pillars 340 may be arranged in a rectangular matrix pattern.

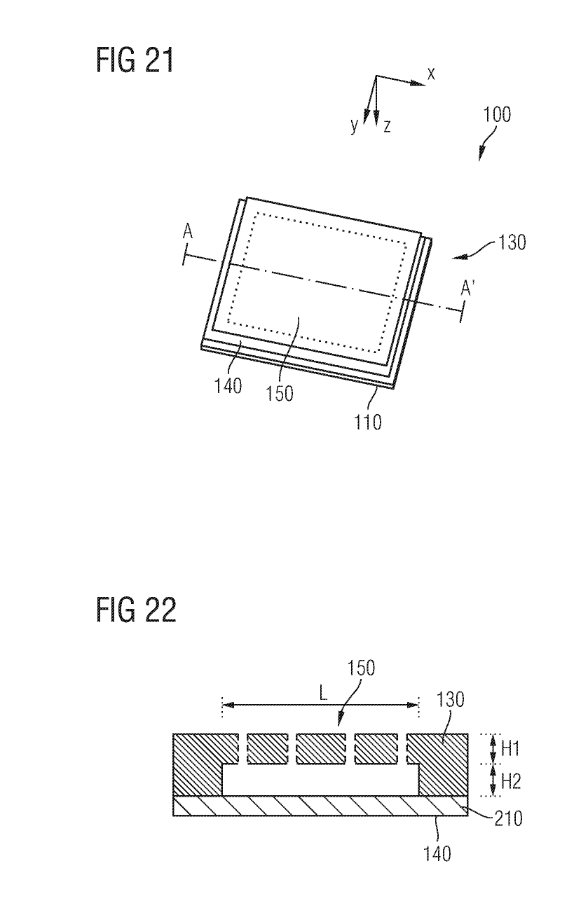

FIG. 25 is a schematic cross-sectional view of a sensor device 300 according to another embodiment.

As shown in FIG. 25, a semiconductor membrane 350 without any pores 152 is formed on a microfiltration device 100 as discussed above. The semiconductor membrane 350 forms the filtrate collection and measurement chamber 325. In the embodiment of FIG. 25, the sensor electrode 220 is part of the filter part 150. In the embodiment of FIG. 26, the sensor electrode 220 is on an inner surface of the semiconductor membrane 350 facing the filter part 150.

FIG. 27 is a schematic plan view of a multisensor device 400 according to an embodiment.

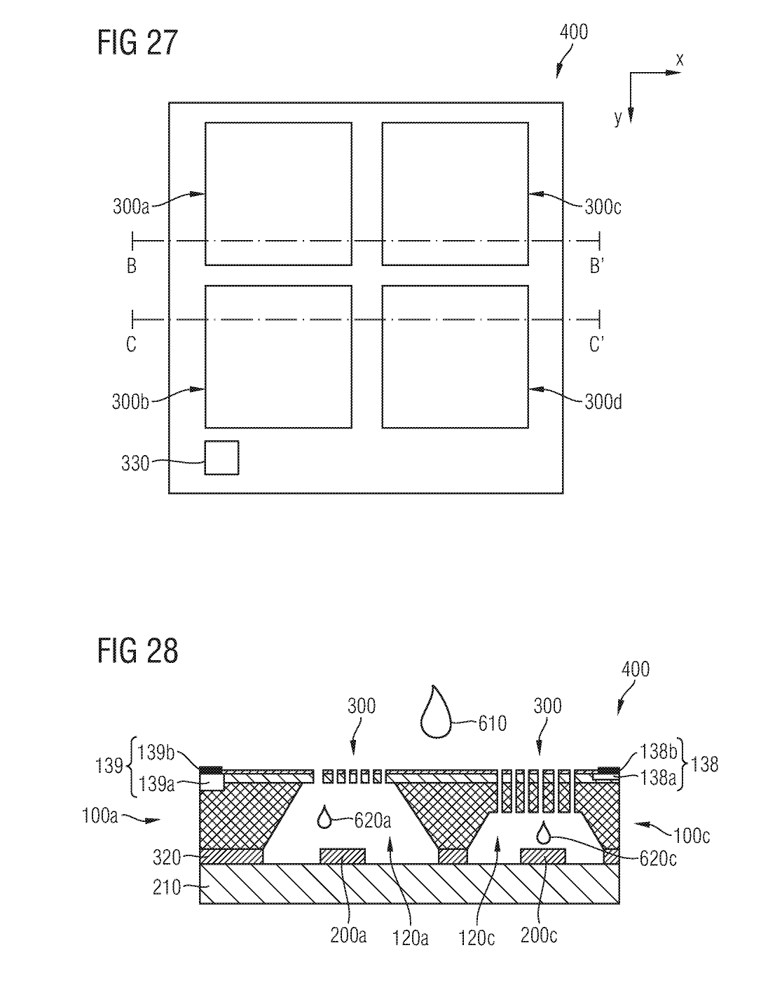

As can be seen from FIG. 27, at least two sensor devices 300a, 300b, 300c and 300d are arranged next to each other in a lateral direction x and a lateral direction y. A cross-sectional view of a portion of the multisensor device 400 taken along the section plane B-B′ of FIG. 27 may typically be shown as in FIG. 28. A cross-sectional view of a portion of the multisensor device 400 taken along the section plane C-C′ of FIG. 27 may typically be shown as in FIG. 29.

The structure of the microfiltration device 100a in FIGS. 27 to 29 is discussed in all detail with regard to FIG. 3H and FIG. 3A to 3G. The structure of the microfiltration device 100c is discussed in all detail with regard to FIG. 4I and FIG. 4A to 4H. However, the contact area 138 for contacting the filter part 150 and the substrate contact area 139 for contacting the substrate 110 may be used in common by the microfiltration devices 100a to 100d.

Although not explicitly shown in FIG. 28, the pores 152 are chosen to have different uniform pore sizes in the sensor devices 300a, 300b, 300c and 300d. Thus, by dripping a feed 610 on the multisensor device 400 as shown in FIG. 27, different filtrates 620a to 620d, depending on the uniform pore sizes of the respective microfiltration devices 100a to 100d are generated and introduced into the respective cavities 120a to 120d. The respective characteristic of the filtrates 620a to 620d is then measured by the sensors 200a to 200d, respectively. Thus a feed 610 such as blood may be separated into different filtrates 620a to 620d to measure different characteristics by the sensors 200a to 200d.

Although in FIG. 27, four sensor devices 300a to 300d are shown being arranged next to each other in a lateral direction or within a lateral plane, it is also possible to assemble a plurality of different sensor devices 300 having filter parts with pores 152 each having different pore sizes.

The bonding 320 is arranged such on the sensor substrate 210, on which the respective sensors 200a to 200d are arranged, that respective cavities 120a to 102d are formed which accommodate the different filtrates 620a to 620d. All of the cavities 120a to 120d may be in fluid contact with the vent-hole 330 to facilitate the introduction of the filtrates 620a to 620d into the respective cavities 120a to 120d, or to equalise the differential pressure across the filter membrane.

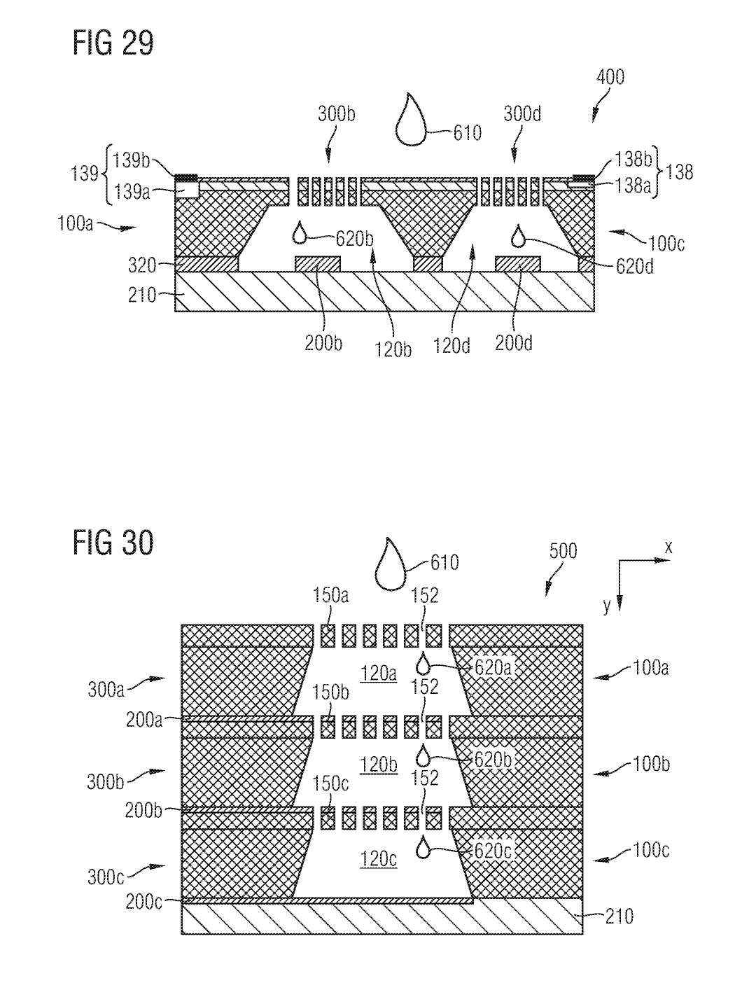

FIG. 30 is a schematic cross-sectional view of a multisensor device 500 according to another embodiment.

As can be seen from FIG. 30, at least two sensor devices 300a to 300c are stacked on each other in a vertical direction z. Although not explicitly shown in FIG. 30, the pores 152 may have different uniform pore sizes. For example, the pores 152 of the microfiltration device 100a may have a first pore size, the pores 152 of the microfiltration device 100b may have a second pore size being smaller than the first pore size, and the pores 152 of the microfiltration device 100c may have a third pore size smaller than the second pore size. Thus, a cascaded multisensor device 500 may be provided, in which a feed 610 is separated into a first filtrate 620a in the cavity 120a of the first sensor device 300a, into a filtrate 620b in the cavity 120b of the sensor device 300b, and into the filtrate 620c in the cavity 120c of the sensor device 300c. In other words, the filtrate 620a is a filtrate from the feed 610 filtered by the filter part 150a, the filtrate 620b is a filtrate from the filtrate 620a filtered by the filter part 150b, and the filtrate 620c is a filtrate from the filtrate 620b filtered by the filter part 150c.

A characteristic of the filtrates 620a to 620c is measured by the sensors 200a to 200c, respectively. As can be seen from FIG. 30, the sensor 200c may be arranged on a sensor substrate 210, wherein the sensor 200c faces the filter part 150c of the sensor device 300c. The sensors 200a and 200b may be part of the filter part 150b and 150c, respectively. As already discussed above, the first layer 332 of a semiconducting or conducting material may constitute the sensor electrode of the sensors 200b and 200c. The filter part 150a comprising a semiconducting or conducting material may be used to introduce the filtrate 620a into the cavity 120a by an electrospray effect as discussed above and as shown in FIG. 33.

Thus, a multisensor device 500 is provided, which uses a microfiltration device 100 made of silicon or epitaxial polysilicon, which may be stacked to form a multi-membrane filter.

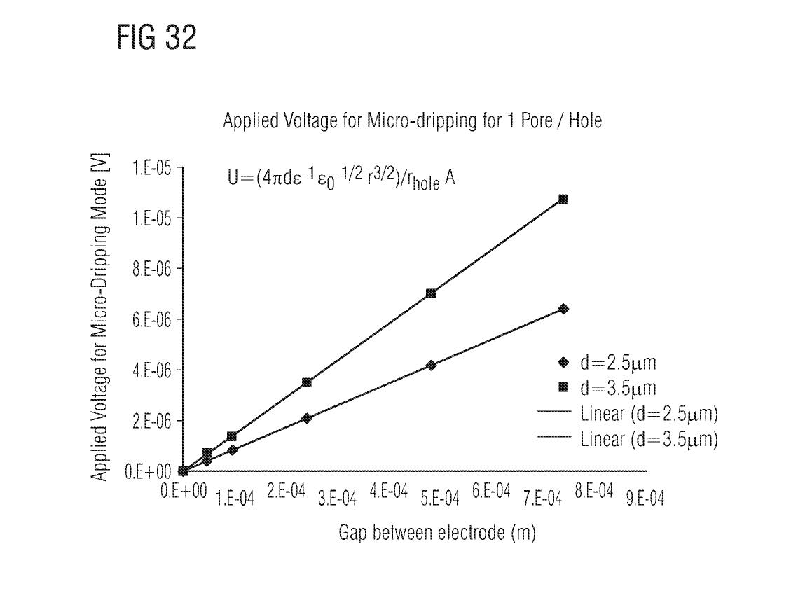

As discussed above, a bio-sensor chip is described to first filter blood and then to measure different blood-related parameters consisting of a MEMS filtration element in combination with a sensor and an ASIC (read-out electronics), for point-of-care testing (POCT). Additionally, the MEMS device allows size-based separation of various blood components like RBCs, WBCs, platelets, for example, as shown in FIG. 32.

FIG. 33A and FIG. 33B are schematic cross-sectional views of a portion of a sensor device 300 according to further embodiments, wherein FIG. 34A and FIG. 34B are plan views of the sensor device 300 according to different embodiments.

As can be seen from FIG. 33A, the first layer 132 of the semiconducting or conducting material can be a contiguous layer, as described above, or can be a patterned layer formed on the second layer 134 of the insulating material.

According to the embodiment of FIG. 33A, the patterned layer of the first layer 132 of the semiconducting or conducting material forms a stacked layer together with the second layer 134 of an insulating material. According to a further embodiment, as can be seen from FIG. 33B, the patterned first layer 132 of the semiconducting or conducting material can be sandwiched between the second layer 134 and the third layer 136 of the insulating material, as described for a contiguous first layer 132 with regard to FIG. 12I, for example. Although FIG. 33B schematically shows the stacked layer of the second layer 134, the first layer 132 and the third layer 136 to be a layer structure, in which the first layer 132 of a conducting or semiconducting material is not electrically insulated from the feed 610 in the reservoir 310, the layer structure as shown in FIG. 33B can also be formed as shown in FIG. 12I, in which the patterned first layer 132 of a semiconducting or conducting material is encapsulated by the second layer 134 and/or the third layer 136 and thus being electrically insulated from the feed 610 or the filtrate 620.

Furthermore, an additional electrode 360 may be applied at the inner wall of the reservoir 310 being in contact with the feed 610. Thus, the additional electrode 360 may be formed as a ring electrode surrounding the inner wall of the reservoir 310. The additional electrode 360 may be used as a counter electrode or a reference electrode for impedance spectroscopy or amperometric measurements.

Examples of different structures of the patterned layer of the first layer 132 of the semiconducting or conducting material are shown as plan views of the sensor device 300 in FIG. 34A and FIG. 34B.

According to FIG. 34A, the patterned layer of the first layer 132 may have an interdigitating electrode structure or a comb electrode structure. In the embodiment as shown in FIG. 34A, the contact area 138 in the frame part 140 being electrically connected to the semiconducting or conducting material in the filter part 150 comprises separate first and second contact areas 1381 and 1382 for contacting different insulated parts constituting first and second electrodes 1321 and 1322 of the first layer 132. Thus, for example, an impedance spectroscopy measurement by determining the transfer function between the interdigitating first and second electrodes 1321, 1322 of the patterned first layer 132 can be performed, wherein the first and second electrodes 1321 and 1322 are contacted by the first contact area 1381 and the second contact area 1382, respectively. For performing an impedance spectroscopy, an insulation of the first layer 132 from the feed 610 and the filtrate 620, as can be seen from the sensor device 300 of FIG. 33B, has the advantage that no electrochemical reaction occurs between the conducting or semiconducting patterned first layer 132 and the feed 610 or the filtrate 620.

However, in case the patterned first layer 132 (or as well the contiguous conducting or semiconducting first layer 132) is used to perform amperometric measurements, the generation of a current from an electrode of the first layer 132 into the feed 610 can be of advantage. In such a case, the structure as shown in FIG. 33A, in which the conducting or semiconducting first layer 132 is in electric contact with the feed 610 may be used.

As can be seen for the embodiment of FIG. 34B, the number of separate electrodes formed by patterning the first layer 132 is not limited to two separate first and second electrodes 1321, 1322 as shown in FIG. 34A. In case of an amperometric measurement, for example, three electrodes 1383 to 1385 may be used wherein a third electrode 1323 is used as a working electrode, the fourth electrode 1324 is used as a reference electrode and the fifth electrode 1325 is used as an auxiliary electrode. As can be seen from FIG. 34B, the third electrode 1323 is connected to a third contact area 1383, the fourth electrode 1324 is connected to a fourth contact area 1384 and the fifth electrode 1325 is connected to a fifth contact area 1385.

The structure of the respective electrodes as described above is not limited to a specific form and may be adapted to the field of application. For example, as can be seen from FIG. 34B, the fifth electrode 1325 may have a meander form, wherein the fourth electrode 1324 may have an antenna form, and the third electrode 1323 may have a contiguous plane form. Further structures of the above described electrodes may comprise a spiral form for inductive coupling or a ring form. In all described embodiments, the first layer 132 may be also not patterned and may be a contiguous electrode covering the complete surface of the second layer 134, wherein, for example, the additional electrode 360 may be used as a counter electrode. Furthermore, the additional electrode 360 may comprise separate electrodes to be separately contacted as a reference electrode and an auxiliary electrode, wherein the first layer 132 being in contact with the feed 610 may be used as the working electrode for amperometric measurements.

Thus, the first to fifth electrodes 1321 to 1325 of the structured or patterned first layer 132 or a contiguous first layer 132 of a semiconducting or conducting material in combination with one or more additional electrodes 360 may be used to perform measurements on the feed 610 and/or the filtrate 620 by means of impedance spectroscopy or amperometric measurements. The first to fifth electrodes 1321 to 1325 within or on the second layer 134 may be structured, to perform measurements on the feed 610 being for example whole blood, wherein the filtrate 620 may be a blood plasma. The above described electrode structures can comprise two electrodes for impedance spectroscopy measurements or three electrodes for amperometric or voltammetric measurements. By further using the sensor electrode 220, additional measurements on the filtrate 620 may be performed. An application of the two separate measurements of the feed 610 and the filtrate 620 may be a detection of hemolysis in the whole blood of the feed 610 before filtering by the filter part 150 and the detecting of potassium concentration of the filtrate 620, i.e. the blood plasma, after filtration by the filter part 150.

The diameter of the structured or patterned first layer 132 constituting the first to fifth electrodes 1321 to 1325 is higher than the pore size of the pores 152 of the filter part 150, in order to prevent an electrical separation of respective parts of the first to fifth electrodes 1321 to 1325 due to the pores 152 extending through the first layer 132 and the second layer 134 and optionally the third layer 136. However, it is also possible that the conductive or semiconductive first layer 132 is covering some of the pores 152 of the second layer 134. In this case, the structure has to be designed such that enough freely exposed pores 152 remain to ensure a sufficent throughput performance of the filter part 150.

In case the pores 152 extend through both the first layer 132 and the second layer 134, and optionally the third layer 136, the first layer 132 may be patterned in a lithographical patterning step in the manufacturing method as described above, in particular between the manufacturing steps of FIGS. 3E and 3F, or between the manufacturing steps of FIGS. 4F and 4G, or between the manufacturing steps of FIGS. 5D and 5E, or between the manufacturing steps of FIGS. 7C and 7D, or between the manufacturing steps of FIGS. 8A and 8E, or the between the manufacturing steps of FIGS. 12A and 12B, or the between the manufacturing steps of FIGS. 13A and 13B. However, the first layer 132 may also patterned after the above manufacturing steps, if it is possible from a manufacturing process view.

Thus, a sensor device 300 may be provided which is adapted to detect the concentration of potassium in the blood plasma constituting the filtrate 620, wherein before the detection of potassium concentration in the filtrated blood, the filtrate 620, the occurrence and the extent of hemolysis can be detected. Such a measurement can be performed directly at the filter part 150 by means of impedance spectroscopy (detection of the destroyed erythrocytes). Due to the electric insulation of the first and second electrodes 1321 and 1322, as can be seen in FIG. 33B, no electrochemical effects can occur, falsifying the impedance measurements. The two frist and second electrodes 1321, 1322 of the sensor device 300 of FIG. 34A can be provided in the patterned first layer 132 of a semiconducting or conducting material as interdigitated electrodes (IDE). In case the potential of the first layer 132 in the filter part 150 shall be defined, the additional electrode 360 may be provided at the inner wall of the reservoir 310. The additional electrode 360 may comprise, for example, an silver chloride (Ag/AgCl)—reference electrode for electrochemically contacting the electrolyte such as the whole blood constituting the feed 610 within the reservoir 310. The conducting or semiconducting first layer 132 on or in the filter part 150 may thus have a patterned structure such as a comb structure or an interdigitated comb structure.

Although specific embodiments have been illustrated and described herein, it will be appreciated by those of ordinary skilled in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A microfiltration device, comprising
a substrate having a first surface and a second surface opposite to the first surface, the substrate including a cavity between the first surface and the second surface, and
a microfilter including a frame part in contact with the substrate and a filter part abutting the cavity, the microfilter comprising in both the frame part and the filter part a semiconducting or conducting material.

2. The microfiltration device of claim 1, wherein the microfilter comprises a first layer of the semiconducting or conducting material.

3. The microfiltration device of claim 2, wherein the microfilter further comprises a second layer of an insulating material.

4. The microfiltration device of claim 3, wherein the first layer of the semiconducting or conducting material is a patterned layer on the second layer of the insulating material.

5. The microfiltration device of claim 3, wherein the microfilter further comprises a third layer of an insulating material, the first layer being sandwiched between the second and the third layer.

6. The microfiltration device of claim 1, wherein the substrate includes an insulating material.

7. The microfiltration device of claim 1, wherein the microfilter and the substrate comprise a same material.

8. The microfiltration device of claim 1, wherein the microfilter is bonded on the substrate.

9. The microfiltration device of claim 1, wherein the semiconducting or conducting material comprises at least one of polysilicon, silicon, porous silicon, and a metal.

10. The microfiltration device of claim 1, wherein the insulating material comprises at least one of an oxide, a nitride, a carbide, and a glass.

11. The microfiltration device of claim 1, wherein the filter part comprises pores arranged in a regular pattern.

12. The microfiltration device of claim 1, wherein the filter part comprises pores of a uniform pore size, the uniform pore size being a size in a range of 1 μm to 10 μm.

13. The microfiltration device of claim 1, further comprising a contact area in the frame part, the contact area being electrically connected to the semiconducting or conducting material in the filter part.

14. A sensor device, comprising:
a microfiltration device, comprising
a substrate having a first surface and a second surface opposite to the first surface, the substrate including a cavity between the first surface and the second surface, and
a microfilter including a frame part in contact with the substrate and a filter part abutting the cavity, the microfilter comprising in both the frame part and the filter part a semiconducting or conducting material; and
a sensor located at a filtrate side of the filter part, the sensor being adapted to measure a characteristic of a filtrate.

15. The sensor device of claim 14, wherein the sensor comprises an optical sensor.

16. The sensor device of claim 14, wherein the sensor comprises a sensor electrode.

17. The sensor device of claim 16, wherein the filter part constitutes the sensor electrode of the sensor.

18. The sensor device of claim 14, wherein the sensor comprises an application-specific integrated circuit.

19. The sensor device of claim 14, wherein the sensor is arranged on a sensor substrate, the sensor substrate being bonded to the microfiltration device such that the sensor faces the filter part.

20. A multisensor device, comprising at least two sensor devices of claim 14 arranged next to each other in a lateral direction, or comprising at least two sensor devices of claim 14 stacked on each other in a vertical direction.
